(19) United States
(12) Patent Application Publication
(10) Pub. No.: US 2006/0014712 A1
(43) Pub. Date: Jan. 19, 2006

(54) Controlled delivery of therapeutic compounds

(75) Inventor: Toomas Neuman, Solana Beach, CA (US)

Correspondence Address: Todd A. Lorenz;Dorsey & Whitney LLP, Intellectual Property Department, 555 California Street, Suite 1000, San Francisco, CA, 94104-1513, US

(73) Assignee: CeMines, Inc.

(21) Appl. No.: 11/141,725

(22) Filed: May 31, 2005

Related U.S. Application Data
(60) Provisional application No. 60/575,660, filed on May 30, 2004.

(51) Int. Cl. A61K 48/00; A61K 38/17

(52) U.S. Cl. 514/44; 514/2

(57) ABSTRACT

The present invention provides compositions for the controlled delivery of compounds into cells. Entry of the compound into a cell is mediated by a cell penetrating peptide capable of translocating the compound across a cell membrane. An inhibitor of cell penetrating peptide, which activity is regulatable by action of a protease, serves to limit delivery of the compound to cells and tissues having the protease activity.

### STATEMENT OF RELATEDNESS

This application claims the benefit of Provisional application Ser. No. 60/575,660 filed May 30, 2004, and is hereby expressly incorporated by reference in its entirety.

### FIELD

The present invention relates to compositions for the controlled delivery of compounds of interest into cells, and more particularly to intracellular transport of therapeutic agents directed against biological molecules acting in the cell nucleus. Delivery of the compounds into cells is controlled by altering the membrane permeability characteristics of the compositions.

### BACKGROUND

Selectivity of a drug is a desirable feature for limiting the adverse side effects from unrestricted exposure to a therapeutic agent and for enhancing the effectiveness of treatment. In addition to designing therapeutic agents with high specificity for the intended molecular target, selectivity may also be achieved by controlled transport through biological barriers and selective activation of the therapeutic agent.

Controlling transport of the therapeutic agent through biological barriers provides one basis for selectively delivering the therapeutic agent to the intended target. Strategies for selective transport include use of a targeting component that directs the agent to a specific cell surface molecule, which is then internalized via regulated cellular transport mechanisms. One method includes use of antibodies selective for a unique cell surface antigen or use of ligands selective for a receptor expressed on the surface of the targeted cell. For example, if a cell population expresses a unique cell surface marker, an antibody can be raised against the unique marker and the therapeutic agent linked to the antibody. Upon administration of the antibody-drug complex, the binding of the antibody to the cell surface marker could result in the delivery of relatively high concentration of the drug to the cell. U.S. Pat. No. 5,527,527 describes use of antibodies against the transferrin receptor while Pardridge, W. M. et al., Pharm. Res. 12: 807-816 (1995) describes use of human insulin receptors for this purpose. Inclusion of antibodies into drug delivery vehicles, such as liposomes, also allows targeting of the drug delivery vehicle to specific cellular targets (see, e.g., U.S. Pat. No. 5,858,382). The same concept applies to use of ligands and homing peptides that bind to cell surface receptors (see, e.g., U.S. Pat. Nos. 5,442,043; 4,902,505; 4,801,575; and 6,576,239). For example, the botulinum neurotoxin heavy chain can target to cholinergic motor neurons and may be used to deliver compounds to these cells (U.S. Pat. No. 6,670,322). Selective targeting approach, however, requires restricted presence of the cell surface marker on the cells being targeted for therapy. General expression of the cell surface antigen or receptor on non-targeted cells makes such targeted delivery less desirable while absence of specific markers on the cell surface severely limit this delivery strategy to only certain types of conditions or diseases.

Another strategy to enhance selectivity of a therapeutic agent is the use of an inactive compound, for example a prodrug, which is converted to the active form by chemical modification. In this approach, endogenous enzymes are exploited to convert the prodrug to the active compound. Endogenous enzyme systems useful in the prodrug strategy include oxidoreductases (e.g., aldehyde oxidase, amino acid oxidase, cytochrome P450 reductase, DT-diaphorase) transferases (e.g., thymidylate synthase, thymidine phosphorylase, glutathione S-transferase), hydrolases (e.g., carboxylesterase, alkaline phosphatase, β-glucuronidase), and lyases. Selectivity is obtained if expression of the endogenous enzyme is restricted to the tissues or cells being targeted for therapy. Variations of this approach include the delivery of non-endogenous enzymes to the target cell via an antibody (“ADEPT” or antibody-dependent enzyme prodrug therapy; U.S. Pat. No. 4,975,278) or introducing the gene encoding the non-endogenous enzyme into the targeted cells (“GDEPT” or gene dependent enzyme-prodrug therapy; see, e.g., Melton, R. G. and Sherwood, R. E., J Natl Cancer Inst. 88(34):153-65. (1996)). Depending on the cells or tissues being targeted, examples of non-endogenous enzymes used for prodrug activation include nitroreductase cytochrome P450, purine-nucleoside phosphorylase, thymidine kinase, alkaline phosphatase, β-glucuronidase, carboxypeptidase, and cytosine deaminase. The advantage of using non-endogenous enzymes is that conversion of the prodrug does not occur except in those cells targeted by the antibody-enzyme complex or in cells modified by introduction of the enzyme-encoding gene. The use of catalytic antibodies as a non-endogenous enzyme has extended this approach for unique prodrug substrates (see, e.g., U.S. Pat. No. 6,702,705). These strategies are effective if the prodrug or activated compound is itself capable of entering the targeted cell. Lack of permeability of the compounds can limit the use of these techniques.

It is desirable to have other approaches for controlled delivery of compounds into cells to augment or provide alternatives for currently known methods. A needed feature, in addition to selectivity, is the ability to deliver a wide variety of compounds, including molecules not normally permeable to the cell membrane.

### SUMMARY OF THE INVENTION

The present invention provides compositions and methods for the controlled delivery of compounds of interest, particularly therapeutic compounds, into target cells. The compositions herein exploit the ability of cell penetrating peptides, once released from inhibition, to translocate compounds attached thereto across cell membranes. In the compositions herein, a cell penetrating peptide inhibitor inhibits the activity of the cell penetrating peptide. The inhibitor's activity is controlled by the presence of a cleavage site in the composition, whereby cleavage at the cleavage site by a cleaving agent disrupts the inhibitor's activity, thereby disinhibiting the cell penetrating peptide and allowing translocation of the cell penetrating peptide and the compound attached thereto across the cell membrane. By using a cleavage site recognized by proteases, particularly extracellular proteases present at and/or proximal to the cells being targeted, delivery of the compounds of interest is generally confined to the target cells.

Accordingly, in one aspect, the present invention provides compositions comprising a cell penetrating peptide, a cell penetrating peptide inhibitor, a compound of interest, such as a reporter molecule or a therapeutic agent, and a cleavage site which when acted upon by a cleaving agent disinhibits the cell penetrating peptide to permit entry of the compound of interest into the target cell. The compositions may further comprise a subcellular localization signal, such as a nuclear localization signal, to direct the compound of interest to a specific intracellular region, thereby increasing the local intracellular concentration of the compound. The subcellular localization signal may also be inhibited by the cell penetrating peptide inhibitor, and disinhibited by the action of a cleaving agent. The use of a nuclear localization signal is advantageous when the therapeutic compounds act on a molecule active in the cell nucleus. In a particularly preferred embodiment, the cell penetrating peptide is modified to include the nuclear localization signal.

The cell penetrating peptides may be based on known peptides, including, but not limited to, penetratins, transportans, membrane signal peptides, and viral proteins, for example Tat protein and VP22 protein, and translocating cationic peptides. Also provided is a novel translocating cationic peptide active for a variety of cells types, where the peptide has the sequence RPKKRKVRRR.

The cell penetrating peptide inhibitors are peptides that mask or interact with the cell penetrating peptide, or otherwise perturb its function in compositions of the invention. Generally, the cell penetrating peptide inhibitors have a loop sequence which turns back to the cell penetrating peptide, interacting or wrapping the cell penetrating peptide, and/or forming a semi-cyclic peptide structure. In one embodiment, the loop sequence comprises one or more beta-turns or beta bends to bring a cell penetrating peptide inhibitor into proximity with a cell penetrating peptide for the purpose of inhibiting the cell penetrating peptide's activity. In another embodiment, the loop sequence comprises flexible loop linkers when the inibitor and cell penetrating peptide have an affinity for each other, as through electrostatic attraction. The flexible loop structure, beta-turn or a beta bend will bring the inhibtor peptide into proximity of the cell penetrating peptide and allow it to mask or associate with the cell penetrating peptide and thereby interfere with its translocation activity. An exemplary inhibitory peptide of this structure has the amino acid sequence TTGGSSPQPLEAP or TTGGSSPQGLEAK. Other peptides of similar structure and activity may be identified by molecular modeling techniques, such as DS Modeling 1.2. In another embodiment, variants of the inhibitory sequences may be obtained by substitutions, insertions, or deletions of the amino acid residues in the exemplary inhibitory peptides. Preferred are conservative substitutions that do not eliminate inhibitory activity.

In the compositions described herein, a cleavage site is used to control the translocation activity of the cell penetrating peptide. In one aspect, the cleavage sites are recognition sites for proteases, particularly extracellular proteases present at and/or proximal to the target cell. These proteases may be present in the extracellular matrix or present on the membrane surface of target or neighbouring cells. Accordingly, in one embodiment, the cleavage sites are sequences recognized by metalloproteases (e.g., MMP2, MMP9, etc.). In another embodiment, the cleavage sites are sequences recognized by cathepsins (e.g., cathepsin B and cathepsin D, etc.). In a further embodiment, the cleavage sites are sequences recognized by trypsins and other proteases that cleave protease activated receptors (PARs).

The cargo, or compounds of interest, may comprise any compound capable of being transported into a cell by the compositions described herein. The compounds of interest are generally not cell permeable, and rely on the translocation activity of an attached cell penetrating peptide for delivery into a target cell. Agents of interest include small organic molecules, such as reporter molecules or therapeutic compounds (e.g., cytotoxic drugs); bioactive peptides and proteins; and nucleic acids. In one embodiment, a single compound may be delivered into the cell. In another embodiment, a plurality of compounds (i.e., a combination of compounds) may be translocated into the target cell. Different compositions may be used to direct delivery of compounds of interest to different cellular targets by the appropriate choice of cleavage sites. In one embodiment, a combination comprising a plurality of compositions having a variety of cleavage sites that direct compounds of interest to a variety of target cells is provided. Additionally, different subcellular localization sequences may be used to direct delivery of compounds of interest to different subcellular sites. In one embodiment, a combination comprising a plurality of compositions having a variety of subcellular localization sequences that direct compounds of interest to a variety of subcellular localizations is provided.

In one aspect, the compounds of interest are peptide modulators of transcription factors. Generally, a peptide mimic competes with a transciption factor for binding to one or more of its natural binding partners.

The compositions are used in methods to deliver compounds of interest, such as therapeutic agents, into target cells. In a preferred embodiment, the compositions are used in methods for the nuclear delivery of therapeutic compounds directed to molecular targets acting in the cell nucleus. The methods generally comprise contacting the target cell with the composition, whereby the composition is capable of being converted to the cell permeable form by a cleaving agent present at and/or proximal to the targeted cell. Preferably, the cells selected for targeted delivery will express the extracellular protease or be localized near extracellular matrix containing the active protease. These methods provide a way of selectively delivering a therapeutic agent for treating a disease condition.

Thus, in one embodiment, the method comprises administering the compositions to a host to treat a condition or disease, whereby the compound is a therapeutic agent. A variety of disorders may be treated by the compositions, including cancer, inflammatory disorders, and allergic responses. Administration may be systematic or localized, depending on the condition and the therapeutic compound. For disorders of the skin, administration via topical solution or via a transdermal patch can localize the therapeutic effect. Thus, in one embodiment, transdermal systems containing the compositions may be used to deliver therapeutic agents to the host, especially to treat disorders of the skin, such as melanoma.

The compositions and methods of the invention provide advantages over other regulated delivery methods. Entry of the compounds is limited to target cells having protease acitivty in their vicinity, which allows use of higher doses of therapeutic agent while minimizing toxicity to healthy cells.

### BRIEF DESCRIPTION OF THE DRAWINGS

FIG. 1 shows effect of peptide inhibitors of transcription factors MITF, SOX10, and STAT3 on activity of Dct/Trp2 promoter in transient CAT assay. The inhibitors are based on amino acid sequences that interact with protein interaction domains on the transcription factors.

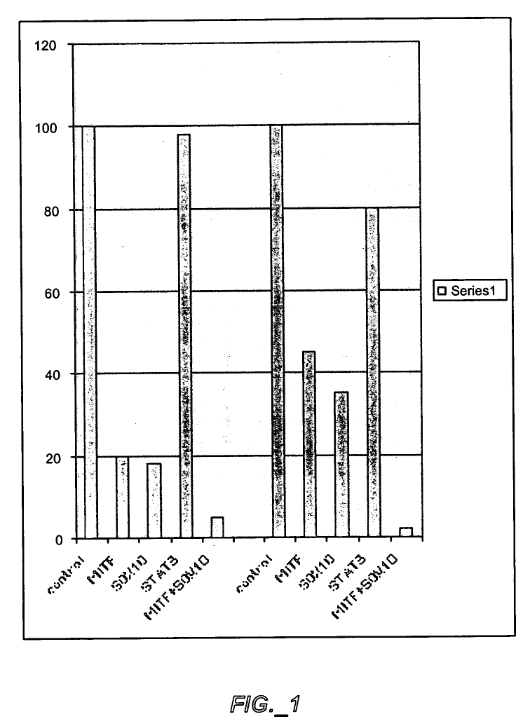

### DETAILED DESCRIPTION

The present invention provides compositions and methods for the controlled delivery of various compounds of interest into cells, particularly the delivery of therapeutic compounds. The compositions use a cell penetrating peptide or translocating peptide to translocate a linked or conjugated compound across the cell membrane. Translocation activity of the cell penetrating peptide is controlled by the presence of an inhibitor of the cell penetrating peptide that reduces membrane penetrating characteristics of the composition. Modification of the composition by cleavage, particularly through use of a cleavage site for an extracellular protease, releases the inhibiting component, thereby allowing translocation of the compound into the cell. This permits translocation of compounds into cells uniquely or preferentially expressing the protease while limiting entry into cells not expressing the proteases. Inclusion of subcellular targeting sequences, either independent of or merged with the cell penetrating peptide, provides an additional element for increasing the specificity of delivery.

Regulated Delivery Compositions and Prodrugs

In accordance with the above, the present invention provides compositions comprising a cell penetrating peptide inhibitor, the cell penetrating peptide, a cargo or compound of interest to be delivered into the cell, and a cleavage site. Optionally, the compositions also include an intracellular (i.e., subcellular) localization signal to direct the compound to specific compartments within the targeted cell.

“Target” cells include any cell being targeted for delivery of the compounds. In particular, the targeted cells have in their vicinity an extracellular protease capable of acting on the cleavage site present on the composition. Such cells include, among others, hyperproliferative cells that are de-differentiated, immortalized, neoplastic, malignant, metastatic or transformed. Examples include, but are not limited to cancer cells such as sarcoma cells, leukemia cells, carcinoma cells, or adenocarcinoma cells. Specified cancer cells include, but are not limited to, breast cancer cells, lung cancer cells, brain cancer cells, hepatoma cells, liver cancer cells, pancreatic carcinoma cells, oesophageal carcinoma cells, bladder cancer cells, gastrointestinal cancer cells, ovarian cancer cells, skin cancer cells, prostate cancer cells, and gastric cancer cells.

In another embodiment, the cells are those involved in the inflammatory process. These include endothelial cells, leukocytes, mast cells, and polymorphonuclear cells taking part in allergic and inflammatory responses. For example, mast cells are known to release the proteases chymase and tryptase in response to binding of IgE to the IgE receptor. In addition, the proteases thrombin and trypsin mediate signal transduction events during the inflammatory response.

It is to be understood that other targeted cells can be identified based on the tissue or the disease condition being treated. The descriptions above of the various cell types are for purposes of illustration and not limitation.

For translocating a cargo into the cells, the compositions comprise a cell-penetrating agent. Cell penetrating agents or translocation agents comprise agents that facilitate delivery of an associated compound of interest or cargo across a cell membrane. It is known that certain peptides have the ability to penetrate a lipid bilayer (e.g., cell membranes) and translocate an attached cargo across the cell membrane. This is referred to herein as “translocation activity”. Without being bound by theory, these membrane penetrating peptides appear to enter the cell, in part, via non-endocytic mechanisms, as indicated by the ability of the cell penetrating peptides to enter the cell at low temperatures (e.g., 4° C.) that would normally inhibit endocytic, receptor-based, internalization pathways. Peptides with cell penetrating properties include, by way of example and not limitation, penetrating, Tat-derived peptides, signal sequences (i.e., membrane translocating sequences), arginine-rich peptides, transportans, amphipathic peptide carriers, and the like (see, e.g., Morris, M. C. et al., Nature Biotechnol. 19:1173-1176 (2001); Dupont, A. J. and Prochiantz, A., CRC Handbook on Cell Penetrating Peptides, Langel, Editor, CRC Press, (2002); Chaloin, L. et al., Biochemistry 36(37):11179-87 (1997); and Lundberg, P. and Langel, U., J. Mol. Recognit. 16(5):227-233 (2003); all publications incorporated herein by reference).

In one embodiment, the cell-penetrating agents are penetrating, as exemplified by peptides derived from the Antennapedia protein. Antennapedia is a homeodomain containing protein composed of three α-helices, with helices 2 and 3 connected by a β-turn. A 16 amino acid sequence RQIKIWFQNRRMKWKK from the third helix is capable of translocating across the cell membrane bilayer and has the ability to translocate compounds attached to the peptide via the lipid penetrating activity of the peptide. Along with the native sequence, variant Antennapedia based peptides with cell penetrating properties have also been described (Derossi, D. et al., Trends Cell Biol. 8:84-87 (1998)), including the retroinverso and D-isomer forms (Brugidou, J. et al., Biochem Biophys Res Commun. 214(2):685-93 (1995)).

In another embodiment, the cell penetrating peptides comprise a membrane signal peptide or membrane translocation sequence capable of translocating across the cell membrane. A cell penetrating “signal peptide” or “signal sequence” refers to a sequence of amino acids generally of a length of about 10 to about 50 or more amino acid residues, many (typically about 55-60%) residues of which are hydrophobic such that they have a hydrophobic, lipid-soluble portion. Generally, a signal peptide is a peptide capable of penetrating through the cell membrane to allow the export of cellular proteins.

Signal peptides can be selected from the SIGPEP database (von Heijne, Protein Sequence Data Analysis 1:4142 (1987); von Heijne and Abrahmsen, L., FEBS Letters 224:439-446 (1989)). Algorithms can also predict signal peptide sequences for use in the compositions (see, e.g., SIGFIND—Signal Peptide Prediction Server version SignalP V2.0b2, accessible at world wide web sites cbs.dtu.dk/services/SignalP-2.0/or world wide web 139.91.72.10/sigfind/sigfind.html). When a specific cell type is to be targeted, a signal peptide used by that cell type can be chosen. For example, signal peptides encoded by a particular oncogene can be selected for use in targeting cells in which the oncogene is expressed. Additionally, signal peptides endogenous to the cell type can be chosen for importing biologically active molecules into that cell type. Any selected signal peptide can be routinely tested for the ability to translocate across the cell membrane of any given cell type (see, e.g., U.S. Pat. No. 5,807,746, incorporated by reference). Exemplary signal peptide sequences with membrane translocation activity include, by way of example and not limitation, those of Karposi fibroblast growth factor AAVALLPAVLLALLAPAAADQNQLMP.

In another embodiment, the cell penetrating peptide sequence comprises the human immunodeficiency virus (HIV) Tat protein, or Tat related protein (Fawell, S. et al., Proc. Natl. Acad. Sci. USA 91:664-668 (1994); Nagahara, H. et al., Nat. Med. 4:1449-1452 (1998); publications incorporated herein by reference). The HIV Tat protein is 86 amino acids long and is composed of three main protein domains: a cystein rich, basic, and integrin-binding regions. Tat binds to the tar region of the HIV genome to stimulate transcription of viral genes via the long terminal repeat (LTR). In addition to the transcriptional stimulating activity, Tat also displays a membrane penetrating activity (Fawell, S. et al., supra). Tat peptides comprising the sequence YGRKKRRQRRR (i.e., amino acid residues 48-60) are sufficient for protein translocating activity. Additionally, branched structures containing multiples copies of Tat sequence RKKRRQRRR (Tung, C. H. et al., Bioorg. Med Chem 10:3609-3614 (2002)) can translocate efficiently across a cell membrane. Variants of Tat peptides capable of acting as a cell penetrating agent are described in Schwarze, S. R. et al., Science 285:1569-1572 (1999).

Another embodiment of cell penetrating agents comprise Herpes Simplex Virus VP22 tegument protein, its analogues and variants (Elliott, G. and O'Hare, P., Gene Ther. 6:12-21 (1999); Derer, W. et al., J. Mol. Med. 77:609-613 (1999)). VP22, encoded by the UL49 gene, is a structural component of the tegument compartment of the HSV virus. A composition containing the C-terminal amino acids 159-301 of HSV VP22 protein is capable of translocating different types of cargoes into cells. Translocating activity is observed with a minimal sequence of DAATATRGRSAASRPTERPRAPARSASRPRRPVE. Homologues of VP22 found in herpes viruses are are also capable of delivery of attached compounds of interest across cell membranes (Harms, J. S. et al., J. Virol. 74:3301-3312 (2000); Dorange, F. et al., J. Gen. Virol. 81:2219-2230 (2000)).

In another embodiment, the cell penetrating peptides comprise cationic peptides with membrane translocation activity. Cationic amino acids include, among others, arginine, lysine, and ornithine. Active peptides with arginine rich sequences are present in the Grb2 binding protein, having the sequence RRWRRWWRRWWRRWRR (Williams, E. J. et al., J. Biol. Chem. 272:22349-22354 (1997)) and polyarginine heptapeptide RRRRRRR (Chen, L. et al., Chem. Biol. 8:1123-1129 (2001); Futaki, S. et al., J. Biol. Chem. 276:5836-5840 (2001); and Rothbard, J. B. et al., Nat. Med. 6(11):1253-7 (2000)). An exemplary cell penetrating peptide of this type has the sequence RPKKRKVRRR, which is found to penetrate the membranes of a variety of cell types. Also useful are branched cationic peptides capable of translocation across membranes, including by way of example and not limitation, (KKKK)₂GGC, (KWKK)₂GCC, and (RWRR)₂GGC (Plank, C. et al., Human Gene Ther. 10:319-332 (1999)).

In a further embodiment, the cell penetrating peptides comprise chimeric sequences of cell penetrating peptides that are capable of translocating across cell membrane. An exemplary molecule of this type is transportan GALFLGFLGGAAGSTMGAWSQPKSKRKV, a chimeric peptide derived from the first twelve amino acids of galanin and a 14 amino acid sequence from mastoporan (Pooga, M et al., Nature Biotechnol. 16:857-861 (1998). Analogues of transportans are described in Soomets, U. et al., Biochim Biophys Acta. 1467(1): 165-76 (2000) and Lindgren, M. et al. Bioconjug Chem. 11 (5):619-26 (2000). An exemplary deletion analogue, transportan-10, has the sequence AGYLLGKINLKALAALAKKIL.

Other types of cell penetrating peptides are the VT5 sequences DPKGDPKGVTVTVTVTVTGKGDPKPD, which is an amphipathic, beta-sheet forming peptide (Oehlke, J., FEBS Lett. 415(2):196-9 (1997); unstructured peptides described in Oehlke J., Biochim Biophys Acta. 1330(1):50-60 (1997); alpha helical amphipatic peptide with the sequence KLALKLALKALKAALKLA (Oehlke, J. et al., Biochim Biophys Acta. 1414(1-2):127-39 (1998); sequences based on murine cell adhesion molecule vascular endothelial cadherin, amino acids 615-632 LLIILRRRIRKQAHAHSK (Elmquist, A. et al., Exp Cell Res. 269(2):237-44 (2001); sequences based on third helix of the islet 1 gene enhancer protein RVIRVWFQNKRCKDKK (Kilk, K. et al., Bioconjug. Chem. 12(6):911-6 (2001)); amphipathic peptide carrier Pep-1 KETWWETWWTEWSQPKKKRKV (Morris, M. C. et al., Nat Biotechnol. 19(12):1173-6 (2001)); and the amino terminal sequence of mouse prion protein MANLGYWLLALFVTMWTDVGLCKKRPKP (Lundberg, P. et al., Biochem. Biophys. Res. Commun. 299(1):85-90 (2002)).

It is to be understood that the cell penetrating peptides may be composed of naturally occurring amino acids or contain at least one or more D-amino acids and amino acid analogues. In another embodiment, the cell penetrating peptides may comprise all D amino acids. As used herein, the term “amino acid” is applicable not only to cell membrane-permeant peptides, but also to peptide inhibitors of cell penetrating peptides, any linker moieties, subcellular localization sequences, and peptide cargos, including peptide pharmaceutical agents (i.e., all the individual components of the present compositions).

The term “amino acid” is used in its broadest sense, and includes naturally occurring amino acids as well as non-naturally occurring amino acids, including amino acid analogs and derivatives. For example, homo-phenylalanine, citrulline, and norleucine are considered amino acids for the purposes of the invention. “Amino acids” also includes imino residues such as proline and hydroxyproline. The side chains may be either the (R) or (S) configuration. If non-naturally occurring side chains are used, non-amino acid substituents may be used.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral transepithelial and transdermal delivery of linked drugs and conjugates, improved bioavailability of membrane-permeant complexes, and prolonged intravascular and interstitial lifetimes when such properties are desirable. The use of D-isomer peptides can also enhance transdermal and oral transepithelial delivery of linked drugs and other cargo molecules. Additionally, D-peptides cannot be processed efficienty for major histocompatibility complex class II-restricted presentation to T helper cells, and are therefore less likely to induce humoral immune responses in the whole organism. Peptide conjugates can therefore be constructed using, for example, D-isomer forms of peptide membrane permeant sequences, L-isomer forms of cleavage sites, and D-isomer forms of bioactive peptides.

In yet a further embodiment, the cell penetrating peptides are retro-inverso peptides. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide may contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C., J. Chem. Soc. Perkin Trans. 1:697-701 (1985); and U.S. Pat. No. 6,261,569. Processes for the solid-phase synthesis of partial retro-inverso peptide analogues have been described (EP 97994-B). All references are incorporated herein by reference.

Generally, the cell penetrating peptides are capable of facilitating transfer of a cargo or compound across a lipid bilayer in a non-selective manner because entry into the cell does not appear to occur by receptor-mediated endocytic pathway. Consequently, the cell penetrating peptide is capable of translocating cargoes non-selectively into a variety of cell types. To control delivery of the compositions into cell types, the compositions further comprise a cell penetrating peptide inhibitor or an inhibitor of cell penetrating peptide. Modification of the inhibitor results in release of the inhibitory effect and formation of an active cell penetrating composition.

The inhibitors of cell penetrating activity comprise any class of molecule capable of inhibiting activity of cell penetrating peptide. The inhibitors may be peptides or proteins that disrupt structure of the cell penetrating peptide, alter the physical characteristics of the compositions as a whole (e.g., hydrophobicity or charge) to alter cell penetrating peptide activity, or mask the cell penetrating peptide activity. Generally, the inhibitors are peptides present adjacent to the cell penetrating peptide, thereby masking or altering its membrane permeability characteristics. However, as will be appreciated by those skilled in the art, the inhibitor may be placed anywhere in the compositions to produce the desired effect, and thus are not limited by being adjacent, directly linked to, or contiguous with the cell penetrating peptide.

In one embodiment, the inhibitor of cell penetrating activity comprises a loop sequence which turns back to the cell penetrating peptide, interacting or wrapping the cell penetrating peptide, and/or forming a semi-cyclic peptide structure. Flexible loop linkers between the cell penetating peptide and the cell penetrating peptide inhibitor are of particular use when the inibitor and cell penetrating peptide have an affinity for each other, as through electrostatic attraction. Such structures have been described recently in the context of the controlled delivery of imaging agents (see Jiang et al., Proc. Nat'l. Acad. Sci., 101:17867-17872, 2004, incorporated herein by reference). Alternatively, beta-turns or beta bends, which are commonly found to link two strands of an anti-parallel beta-sheet to form a beta-hairpin structure, may be used to bring a cell penetrating peptide inhibitor into proximity with a cell penetrating peptide for the purpose of inhibiting the cell penetrating peptide's activity. Beta-turns can be classified according to the number of residues in the loop, and by far the most common is the two residue turn, followed in frequency by three, four and five residue turns (see, e.g., Sibanda, B. L. and Thornton, J. M., Nature 316:6024, 170-174 (1985)). In one aspect, the loop or turn may occur through the presence of a glycine and/or proline residues, both of which are found with high frequency in beta bends. Gly residues are conformationally more flexible since its R group has the least amount of steric hindrance, while proline has a fixed Cα-N bond angle because of the ring structure, thereby promoting sharp bends in protein structure. Other sequences suitable for forming beta bends may be determined using molecular modeling programs, such as BTPRED (Shepherd, A. J. et al., Protein Sci. 8(5):1045-55 (1999)) or predicted from naturally occurring beta turn sequences (Wilmot, C. M. and Thornton, J. M., J. Mol. Biol., 203(1):221-232 (1988); Sibanda, B. L. and Thornton, J. M., Nature, 316(6024):170-174 (1985); and Ramirez-Alvarado, M. et al., J. Mol. Biol., 273(4):898-912 (1997).

In another embodiment, the beta turn, beta bend, or loop structures may be based on peptide mimetics. Peptide mimetics are structures which serve as substitutes for peptides or portions of peptides (for review, see Morgan et al., Ann. Reports Med. Chem. 24:243-252 (1989)). Peptide mimetics, as used herein, include synthetic structures that may or may not contain amino acids and/or peptide bonds, but retain the structural and functional features of a core or hybrid polypeptide. Beta-turn mimetics or mimicks of loop structures are described in Kee, K. S. and Jois, S. D., Curr. Pharm. Des. 9(15):1209-24 (2003); Nakanishi, H. et al., Proc Natl Acad Sci USA. 1:89(5):1705-9 (1992); Kahn, M. et al., J Mol Recognit. 1(2):75-9 (1988); U.S. Pat. No. 5,674,976).

In addition to the loop or beta bend, the inhibitory peptide has a sequence which perturbs function of the cell penetrating peptide. A sequence with such activity has the prototypical amino acid sequence TTGGSSPQPLEAP, which inhibits activity of cell penetrating peptide RPKKRKVRRR. Its derivative TTGGSSPQGLEAK, containing a recognition sequence for matrix metalloprotease MMP2 and MMP9 (underlined), also displays inhibitory activity. Similar structural motifs capable of inhibiting cell permeability may be found by molecular modeling analysis of other sequences, such as by use of algorithms used in DS Modeling 1.2 (Discovery Studio Package).

Other functional variants of the inhibitory sequence TTGGSSPQPLEAP or TTGGSSPQGLEAK may be made using art known techniques. A functional variant or functional polypeptide refers to a peptide which posseses the biological function or activity identified through a defined functional assay, and which is associated with a particular biologic activity (i.e., inhibition of cell penetrating peptide). In one embodiment, the variants are substitutional changes of one or more residues to the prototypical inhibitor sequence, where the changes are made in accordance with the following:

TABLE I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Tyr, Met, Leu |
| Ser | Thr |
| Thr | Ser |
| Trp | Tyr, Phe |
| Tyr | Trp, Phe |
| Val | Ile, Leu |

In one embodiment, the inhibitory peptides are conservative variants of the exemplary inhibitor sequence above. Conservative variants as used herein refer to the replacement of an amino acid by another chemically and biologically similar residue. Examples of conservative variations include the substitution of one hydrophobic residue such as isoleucine, valine, leucine, or methionine for another; the substitution of one polar residue for another polar residue, such as substitution of one arginine for lysine, glutamic acid for aspartic acid, or glutamine for asparagines; and the substitution of one hydroxylated amino acid serine or threonine for another.

In other embodiments, the changes are deletions or insertions of a few residues, more preferably one residue to preserve the desired biological activity. Amino acids may be added to the amino or carboxy terminus. Biological activity is readily tested by synthesizing the subsitution, insertion, or deletion variants of the inhibitor and attaching it to different cell penetrating peptides. A detectable cargo, such as a peptide with a reporter molecule (e.g., a fluorescent compound) is coupled to the cell penetrating peptide and examined for its ability to be transported into various cell types. FACS analysis provides a rapid method to detect variants with inhibiting activity. Sites critical for inhibitory function may be determined for the purposes of identifying other peptides with similar activity.

Activation of cell penetrating peptide activity is mediated by chemical transformation (i.e., modification) of the inhibitor component, unmasking or releasing the inhibitory effect of the inhibitor on cell penetrating peptide activity. Generally, the modification is a cleavage reaction mediated by a cleaving agent, which removes the inhibitor, or a portion thereof, from the composition. Typically, the cleavage agent is a protease present in the organism being treated, and more particularly, present on the cells being targeted. A person skilled in the art will appreciate that the compositions of the present invention can be tailored in such a manner that the desired compound of interest is released by action of the protease known to be active in the condition or disease being treated.

Accordingly, in one embodiment, the cell penetrating peptide is attached, linked, or conjugated to the inhibitory component by a suitable cleavage site acted on by a protease. Proteases are divided into two broad categories on the basis of type of attack on the protein: they are exo- and endo-. Proteinases or endopeptidases attack inside the protein to produce large peptides. Peptidases or exopeptidases attack ends or fragments of protein to produce small peptides and amino acids. Proteinases are further divided into additional groups of serine, threonine, cysteine (thiol), aspartic (acid), metallo and mixed depending on the principal amino acid participating in catalysis. The serine, threonine and cysteine peptidases utilize the catalytic part of an amino acid as a nucleophile and form an acyl intermediate; these peptidases can also readily act as transferases. In the case of aspartic and metallopeptidases, the nucleophile is an activated water molecule. For the most part, cleavage sites in the compositions will typically be sequences recognized by endopeptidases. Sequences functioning as substrates for the proteases are readily determined by sequencing of hydrolytic products of natural substrates, consensus sequences obtained from examination of a number of known substrate sites, and testing in model substrates. For example, fluorogenic peptide substrates have been a very powerful tool for determining protease specificity. Another screening technique uses phage display where a cleavable peptide sequence is inserted between a histidine tag affinity anchor and the M13 phage coat protein, pill. Bacteriophages containing preferred peptide recognition sequences for a given protease are cleaved from the resin, recovered, and amplified, whereas the uncleaved phage remain bound to the Ni(II) resin. After several rounds of cleavage and subsequent amplification of the phage, the phagemid DNA plasmids can be sequenced and analyzed for protease substrate specificity preferences. These and other methods known in the art may be used to identify cleavage sequences useful in the present compositions.

In one aspect, the cleavage site comprises substrate for an extracellular endoprotease, particularly an extracellular protease specific to the cells to which the composition is directed. The extracellular protease may be present on the cell surface or is secreted by the cell and/or neighbouring cells, and/or localized to the extracellular matrix (ECM) or basement membrane (BM). Thus the protease is typically present proximal to the targeted cell. In one embodiment, the cleavage site is an amino sequence cleaved by metalloproteinases, a family of multidomain zinc endopeptidases which contain a catalytic domain with a common metzincin-like topology and are responsible for proteolytic events in the extracellular milieu. Metalloproteases are expressed by a variety of cell types and in certain disease conditions, and display broad substrate specificities for a variety of ECM/BM components, such as collagen types I, II, III and IV, laminin and fibronectin. Five major groups of known MMPs include gelatinases, collagenases, stromelysins, membrane-type MMPs, and matrilysins. The activities of MMPs in normal tissue are strictly regulated by a series of complicated zymogen activation processes and inhibition by protein tissue inhibitors for matrix metalloproteinases (“TIMPs”) (Nagase, H., Biochim. Biophys. Acta 1477, 267-283 (2000); Westermarck, J. and Kahari, V. M., FASEB J. 13, 781-792 (1999)). Excessive MMP activity has been implicated in cancer growth, tumor metastasis, angiogenesis in tumors, arthritis and connective tissue diseases, cardiovascular disease, inflammation, and autoimmune diseases (Massova, I. et al., FASEB J. 12:1075 (1998)). For example, increased levels of human gelatinases MMP-2 and MMP-9 activity have been implicated in the process of tumor metastasis (see, e.g., Pyke, C. et al., Cancer Res. 52, 1336-1341 (1992); Dumas, V. et al., Anticancer Res. 19:2929-2938 (1999)).

Accordingly, in one embodiment, the cleavage site is the amino acid sequence for a substrate recognized by a matrix metalloproteinase. MMP-1 (collagenase, interstitial collagenase) recognizes the sequence Pro-Leu-Gly-Leu-Trp-Ala-Arg, and active variants thereof (McGeehan, G. M. et al., J. Biol. Chem. 269(52):32814-32820; Ohkubo, S. et al., Biochem. Biophys. Res, Comm. 266:308-313 (1994)) or shorter sequences Arg-Pro-Gly-Leu, Gly-Ile-Ala, or Gly-Leu-Ala (Miller, E. J., Biochemistry 15(4):787-92 (1976)). MMP-2 (gelatinase A, type IV collagenase) recognizes the sequence Pro-Gln-Gly-Ile-Ala-Gly-Gln (UCL/HGNC/HUGO Human Gene Nomenclature Database). MMP-3 (stromelysin, transin-1) recognizes a sequence P4-P3-P2-P1-P1′-P2′-P3 where P1′, P2′ and P3′ are hydrophobic residues (UCL/HGCN/HUGO Human Gene Nomeclature Database). MMP-7 (matrilysin, uterine metalloproteinase) recognizes the sequence Arg-Pro-Leu-Ala-Leu-Trp-Arg-Ser. MMP-8 (collagenase-2) recognizes the sequence Pro-Leu-Ala-Tyr-Trp-Ala-Arg. MMP-9 (gelatinase B) recognizes the cleavage site Pro-Ley-Gly-Leu-Trp-Ala-Arg, and active variants thereof (McGeehan, G. M. et al., J. Biol. Chem. 269(52):32814-32820 (1994)). MMP-13 (collagenase 3) recognizes the sequence Pro-Leu-Ala-Cys-Trp-Ala-Arg.

Another family of extracellular protease cleavage sites are those recognized by cathepsins, a family of cysteine proteases capable of degrading several ECM components including collagen IV, fibronectin, and laminin. Cathepsins B and C are up-regulated during prostate cancer cell progression and are frequently co-expressed early in the development of prostate cancer. In another example, Cathepsin D is present in increased levels in inflammatory bowel disease and is believed to participate in inflicting mucosal damage in Crohn's disease. High levels of Cathepsin D is found in breast cancer and is associated with tumor reoccurrence and morbidity (Tandon, A. K. et al., N. Engl. J. Med. 322(5):297-302 (1990)). Thus, embodiments of the cleavage sites are amino acid sequences recognized by cathepsins. Cathepsin B displays broad specificity, with preferential recognition sequence Arg-Arg-Xaa. Cathepsin D recognizes the amino acid sequence 1-Phe-Val-2, 4-Gln-His-5, 13-Glu-Ala-14, 14-Ala-Leu-15, 15-Leu-Tyr-16, 16-Tyr-Leu-17, 23-Gly-Phe-24, 24-Phe-Phe-25, and 25-Phe-Tyr-26 bonds in the B chain of insulin.

In another embodiment, the cleavage sites are those recognized by serine proteases, including kallikrein, trypsin, tryptase, and chymase. Exemplary kallikrein protease recognition sequences include, by way of example and not limitation, that of kallikrein 2, which recognizes the substrate P4-P3-P2-Arg-Ser-P2′-P3′ (Cloutier, S. M. et al., Eur. J. Biochem. 269:2747-2754 (2002)); prostate specific antigen, a kallikrein serine protease with specificity for substrate Ser-Ser-(Tyr/Phe)-Tyr (Coombs, G. S. et al., Chem Biol. 5(9):475-88 (1998)); and Granzyme B, involved in activation of apoptotic pathway, recognizing the sequence Ile-Glu-Xaa-Asp-Xaa-Gly, and active variants thereof (Harris, J. L. et al., J. Biol. Chem. 273(42):27364-27373 (1998)).

Another serine protease found in human prostatic cancer cells is membrane-type serine protease 1 (MT-SP1). MT-SP1 is predicted to be a modular, type II transmembrane protein that contains a signal/anchor domain, two complement factor 1R-urchin embryonic growth factor-bone morphogenetic protein domains, four low density lipoprotein receptor repeats, and a serine protease domain. Preferential expression of the protease occurs in the gastrointestinal tract and the prostate. The preferred cleavage sequence is (P4(Arg/Lys)-P3-P2(Xaa)-Ser-Arg-P2(Ala) and sequence (P4-(Xaa)P3-(Arg/Lys)P2-(Ser)P1(Arg) P1′(Ala)), where Xaa is a non-basic amino acid (Takeuchi, T., J. Biol. Chem. 275(34):26333-26342 (2000)).

In yet a further embodiment, the cleavage site is an amino acid sequence recognized by calcium-dependent serine endoproteases, such as Furin, which is one member of proprotein convertases that process latent precursor proteins into their biologically active products. Some of its natural substrates include proparathyroid hormone, transforming growth factor β1 precursor, proalbumin, pro-β-secretase, membrane type-1 matrix metalloproteinase, β subunit of pro-nerve growth factor and von Willebrand factor. It is also thought to be one of the proteases responsible for the activation of HIV envelope glycoproteins gp160 and gp140. Amino acid sequence recognized by furin has the sequence R-X-X-R, where X before the second Arg may be Lys, Arg, or Pro (Matthews, G. L. et al., Protein Sci. 3(8):1197-205 (1994)).

Another embodiment of a cleavage site comprises amino acid sequences recognized by serine protease thrombin. Thrombin is a key component in the activation of platelets via proteolysis of fibrinogen but is also involved in mediating inflammatory responses. Cleavage sites for thrombin typically comprise the sequence P4-P3-P2-P1-P1′-P2′-P3, where P1 is preferentially Arg and P2 and P1′ is Gly. If hydrophobic residues are in P4 and P3 positions, P2 is preferably Pro, P1 is preferably Arg, and P1′ and P2′ are preferably non-acidic amino acids (Keil, B., Specificity of Proteolysis, and p. 335. Springer-Verlag Berlin-Heidelberg-New York, (1992)). Thrombin protease cleavage sequences may also be based on thrombin protease sites present in protease activated receptors (PAR), of which four types have been identified. PAR-1 is cleaved at the amino acid sequence Leu-Pro-Asp-Arg-Ser-Phe-Leu-Leu-Arg-Asn; PAR-3 is cleaved at the amino acid sequence Leu-Pro-Ile-Lys-Thr-Phe-Arg-Gly. PAR proteins have addition proteases sites for plasmin, granzyme A, and cathepsin G, which may also be used (see, e.g., Dery, O. et al., Am. J. Physiol. 274(6 Pt 1):C1429-52 (1998)).

In yet a further embodiment, the cleavages sites comprise amino acid sequences recognized by mast cell associated proteases chymase and tryptase. Chymase is a chymotrypsin-like serine protease expressed exclusively in mast cells (MCs), where the protease is stored within the secretary granules and released along with tryptase, heparin, and histamine in response to allergen challenge or other stimuli. Chymase is believed to function in induction of microvascular leakage, inflammatory cell accumulation, neutrophil and lymphocyte chemotaxis, extracellular matrix degradation, and cytokine metabolism. Human α-chymase cleaves the amino acid sequence Asp-Ala-Val-Tyr-Ile/Val-His-Pro-Phe-His-Leu, and variants thereof (see, e.g., Urata, H. et al., J. Biol. Chem. 265(36):22348-22357 (1990)). Tryptase is also a granule-associated serine proteinase that may be involved in causing asthma and other allergic and inflammatory disorders. This protease preferentially cleaves peptide substrates carboxy-terminal to arginine and lysine residues (Kam, C. M. et al., Arch. Biochem. Biophys. 316, 808-814 (1995). PAR-2 is cleaved at the amino acid sequence Ser-Lys-Gly-Arg-Ser-Leu-Ile-Gly-Arg by tryptase. Tryptase is also known to cleave fibronogen, fibronectin, kininogen, and stromelysin.

Although the clevage sites may be separate from other elements of the compositions (e.g., inhibitor of cell penetrating peptide, the cell penetrating peptide, subcellular localization signal, and compound of interest), in other embodiments, the cleavage sites are merged (i.e., integral) with the cell penetrating peptide, analogous to merging of cell penetrating peptide and intracellular localization signal described herein. As noted above, an exemplary sequence combining a protease recognition site and an inhibitor of cell penetrating peptide activity has the sequence TTGGSSPQGLEAK, where the underlined sequence is a clevage site for matrix metalloproteases MMP2 and MMP-9.

As will be appreciated in the art, the specific embodiments described above are not to limit the types of cleavage sites useful in the compositions. Other amino acid sequences acting as substrates for other proteases may be used for controlled delivery of the compounds of interest into cells and will be apparent to those of ordinary skill in the art (see, e.g., Barrett, A. et al, Handbook of Proteolytic Enzymes, Academic Press (1998); incorporated herein by reference). Moreover, it is to be understood that the cleavage sites need not be restricted to proteases expressed by the cells being targeted for delivery of compounds. Non-endogenous proteases may be added to the target cell by an antibody (“ADEPT” or antibody-dependent enzyme prodrug therapy directed to a cell surface antigen (see, e.g., U.S. Pat. No. 4,975,278, incorporated herein by reference) or through use of gene targeting approach (“GDEPT” or gene dependent enzyme-prodrug therapy; U.S. Pat. No. 6,410,328, incorporated herein by reference). This greatly expands the types of cells that can be targeted by the present compositions.

To increase the specificity of delivery, the compositions may further comprise an intracellular targeting or subcellular localization signal to target the compounds of interest to specific subcellular compartments and/or organelles. Subcellular locations include Golgi, nucleus, nuclear membrane, mitochondria, secretory vesicles, and cell membrane. The intracellular targeting domains may be separate or distinctive from the cell penetrating peptide or a therapeutic peptide. By distinctive or separate refers to a subcellular targeting activity not associated with other activities or functions present in the composition. In other embodiments, the intracellular targeting activity is coincident with other activities, such as cell penetrating activity and intracellular targeting activity. In a particularly preferred embodiment, the nuclear loczalization sequences are merged with the cell penetrating peptide activity. That is, the peptide displaying the cell penetrating activity also has an integral nuclear localization activity.

Various intracellular targeting sequences may be used in the compositions. Lysosomal targeting sequences, include, among others, those of Lamp-2 sequence KFERQ (Dice, J. F. et al., Ann. N.Y. Acad. Sci. 674: 58-64 (1992)); Lamp-1 sequence MLIPIAGFFALAGLVLIVLIAYLIGRKRSHAGYQTI (Uthayakumar, S. et al., Cell. Mol. Biol. Res. 41: 405-20 (1995)); or Lamp-2 sequence LVPIAVGMLAGVLILVLLAYFIGLKHHHAGYEQF (Konecki, D. S. et al., Biochem. Biophys. Res. Comm. 205: 1-5 (1994)).

Mitrochondrial targeting sequences include, among others, mitochondrial matrix sequences MLRTSSLFTRRVQPSLFSRNILRLQST of yeast alcohol dehydrogenase III (Schatz, G., Eur. J. Biochem. 165: 1-6 (1987)); mitochondrial inner membrane sequence MLSLRQSIRFFKPATRTLCSSRYLL of yeast cytochrome c oxidase subunit IV (Schatz, supra); mitochondrial intermembrane space sequence MFSMLSKRWAQRTLSKSFYSTATGAASKSGKLTQKLVTAGVAAAGITASTLLYADSLTA of yeast cytochrome EAMTA (Schatz, supra); or mitochondrial outer membrane sequence MKSFITRNKTAILATVMTGTAIGAYYYYNQLQQQQQRGKK of yeast 70 kD outer membrane protein (Schatz, supra).

The subcellular localization sequences may also be endoplasmic reticulum targeting sequences, including the calreticulin sequence KDEL (Pelham, H. R., Royal Society London Transactions B:1-10 (1992)) or adenovirus E3/19K protein sequence LYLSRRSFIDEKKMP (Jackson, M. R. et al. EMBO J. 9: 3153-62 (1990)).

In another embodiment, the subcellular targeting sequence is a nuclear localization sequence (NLS). Generally, nuclear localization sequences are characterized by a short single cluster of basic amino acids (monopartite) or two clusters of basic amino acids separated by a 10-12 amino acid linking region (bipartite structure) and functions to direct the entire protein in which they occur to the cell's nucleus. NLS amino acid sequences used in the art include those from SV40 large T Antigen, with the sequence PKKRKV (Kalderon et al., Cell 39:499-509 (1984)); the human retinoic acid receptor β-nuclear localization signal sequence ARRRRP; the NF.kappa-β p50 associated sequence EEVQRKRQKL (Ghosh et al., Cell 62:1019 (1990)); and NF.kappa.B p65 associated sequence EEKRKRTYE (Nolan et al., Cell 64:961 (1991)). Bipartite nuclear localization activity are described in Boulikas, J. Cell. Biochem. 55(1):32-58 (1994), Dingwall, et al., J. Cell Biol. 107:641-849 (1988) (e.g., double basic NLS's exemplified by nucleoplasmin associated sequence KRPMTKKAGQAKKKK), Kalderon, D. et al., Cell 39:499-509 (1984), and Robbins, J. et al., Cell 64:615-623 (1991). All publications hereby incorporated by reference.

Other types of nuclear localization signals may be identified based on structure and physical properties of each individual amino acid in a sequence (Conti, E. et al., J. Cell 94:193-204 (1998); Conti, E. and Kuriyan, J. Structure Fold Des. 8:329-338 (2000); Hodel, M. R. et al., J. Biol. Chem. 276:1317-1325 (2001); all publications incorporated herein by reference). As described in the art, coupling of an NLSs onto reporter proteins, peptides, or other cargoes not normally targeted to the cell nucleus cause these cargoes to be concentrated in the nucleus (e.g., Dingwall and Laskey, Ann, Rev. Cell Biol. 2:367-390 (1986); Bonnerot, et al., Proc. Natl. Acad. Sci. USA 84:6795-6799, (1987); and Galileo, et al., Proc. Natl. Acad. Sci. USA 87:458462 (1990)).

Embodiments of nuclear localization sequences associated with multiple biological activities, particularly the characteristic of cell penetrating activity, include the sequence PKKKRKVEDPYC (Zanta, Mass. et al., Proc. Natl. Acad. Sci. USA 96:91-96 (1998)). Some sequences, such as the cell penetrating peptide from Antennapedia do not have the classical nuclear localization signal but may accumulate in the nucleus because of affinity of the peptide for DNA. A specific embodiment with a combined cell penetrating and nuclear localization activity has the amino acid sequence RPKKRKVRRR.

In addition to the specific sequences described above, suitable nuclear localization sequence can be obtained from various databases or predicted by use of molecular modeling algorithms (see, e.g., Nair. R. and Rost, B. Nucleic Acids Res. 31(13):3337-33340 (2003); Cokol, M. et al., EMBO Rep. 1(5):411-415 (2000); and Pointing, C. P. et al., Nucleic Acids Res. 27(1):229-232 (1999), all of which provides a compendium of nuclear localization sequences, either experimentally verified or obtained through searches of sequence database). LOC3D available at world wide web site cubic.bioc.Columbia.edu/db/LOC3d/ is an updated database for predictions of sub-cellular localization signals for eukaryotic proteins. Predictions are based on use of four different methods: (i) PredictNLS, which identifies putative nuclear proteins through presence of nuclear localization signals, (ii) LOChom, which identifies nuclear localization signals based on sequence homology, (iii) LOCkey, which infers localization through automatic text analysis of SWISS-PROT keywords, (iv) LOC3Dini, an ab initio prediction based on neural networks and vector support machines.

In another aspect, a regulator of nuclear localization may be used to control or affect nuclear localization activity. The regulatory region modulates transport of the composition having the nuclear localization signal. In one embodiment, the localization regulatory region is a phosphorylation sequence, which is substrate for a cellular kinase. When the sequence is present adjacent to the nuclear localization sequence, phosphorylation decreases import into the nucleus. It is suggested that the phosphorylation masks structural features of the nuclear localization sequence and affects interaction with the nuclear import machinery. These modification sites may be present in various positions relative to the NLS, and thus may exist within, adjacent to, or distant from the nuclear localization sequence. Various phosphorylation sequences based on known substrates for kinases may be used (see, e.g., Harreman, M. T. et al., J. Biol. Chem. March 3 epublication (2004)).

Compounds of Interest and Cargo

The cell penetrating agent is used to deliver a compound of interest or a cargo into a target cell. In a particularly preferred embodiment, a cell penetrating peptide and an associated nuclear localization signal is used to deliver compounds of interest into the target cell nucleus. A compound of interest or a cargo comprises various chemical classes that are capable of being transported into the cell by the compositions described herein. These include, among others, small organic molecules, macrocylic compounds, nucleotides, nucleic acids, peptides, proteins, and carbohydrates.

Small Organic Molecules

In one aspect, the compounds of interest comprise small organic molecules. As used herein, small organic molecules refers to molecules of about 200 to about 2500 daltons, although it may be larger depending on the compound. The organic compounds typically comprise functional groups, for interacting covalently or non-covalently with biological molecules. Functional groups include amines, carbonyl, hydroxyl, or carboxyl groups. The organic compounds often comprise cyclical carbon or heterocyclic structures, and/aromatic or polyaromatic structure substituted with one or more functional groups. Such compounds may be antibiotics, small organic molecule drugs, nucleotides, amino acids, saccharides, fatty acids, steroids, dye molecules (see, e.g., Conn's Biological Stains, 10^{th} Ed. (Horobin, R. W. and Kiernan, J. A.), BIOS Scientific Publishers, Oxford, UK (2002), incorporated herein by reference), and derivatives thereof. Small organic molecules also encompass haptens recognized by antibodies or other proteins, and include, by way of example and not limitation, digoxigenin, dinitrophenol, biotin, oestradiol, fluorescein isothiocyanate (FITC), 3-nitro-4-hydroxy-5-iodophenylacetic acid (NIP), and the like.

A wide variety of compounds of interest, including bioactive compounds, flurochromes, dyes, metals and metal chelates may be delivered into the cell, particularly into the nucleus of the cell, by use of the compositions described herein. Bioactive refers to a compound having a physiological effect on the cell as compared to a cell not exposed to the compound. A physiological effect is a change in a biological process, including, by way of example and not limitation, DNA replication and repair, recombination, transcription, translation, secretion, membrane turnover, cell adhesion, signal transduction, and the like. A bioactive compound includes pharmaceutical compounds.

Bioactive compounds suitable for delivery by the compositions herein, include, among others, chemotherapeutic compounds, including by way of example and not limitation, vinblastine, bleomycin, taxol, cis-platin, adriamycin, and mitomycin. Exemplary chemotherapeutic agents suitable for the present purposes are compounds acting on DNA synthesis and stability. For example, antineplastic agents of the anthracyclin class of compounds act by causing strand breaks in the DNA and are used as standard therapy against cancer. Exemplary anti-neoplastic agents of this class are daunorubicin and doxorubicin. Coupling of these compounds to peptides and proteins are described in Langer, M. et al., J. Med. Chem. 44(9):1341-1348 (2001) and King, H. D. et al., Bioconjug. Chem. 10:279-288 (1999). By attaching or linking the antineoplastic agents to a cell penetrating peptide, the compounds can be translocated into the cell upon cleavage of the inhibitor of cell penetrating activity. Inclusion of a nuclear localization signal further increases the specificity of the compound to the cell nucleus, where these antineoplastics agents typically function.

Other classes of antitumor agents are the enediyne family of antibiotics, representative members of which include calicheamicins, neocarzinostatin, esperamincins, dynemicins, kedarcidin, and maduropeptin (see, e.g., Smith, A. L. and Nicolaou, K. C., J. Med. Chem. 39:2103-2117 (1996)). Similar to doxorubicin and daunorubicin, the antitumor activity of these agents resides in their ability to create strand breaks in the cellular DNA. Conjugates to antibodies have been used to deliver these molecules into those tumor cells expressing antigens recognized by the antibody and shown to have potent antitumor activity with reduced toxicity as compared to the unconjugated compounds (Hinman, L. M. et al., Cancer Res. 53:3336-3342 (1993)). Conjugating the enediyne compounds to the compositions described herein provides another method of regulated delivery of the therapeutic agents into disease cells.

In a further embodiment, the compounds are small molecule modulators of telomerase activity. These include, by way of example and not limitation, alterperynol, a fungal metabolite capable of inhibiting telomerase activity (Togashi, K. et al., Oncol. Res. 10:449-453 ((1998)); isothiazolone derivatives (Hayakawa, N. et al., Biochemistry 38:11501-11507 (1999)); rhodacyanine derivatives (Naasani, I. et al., Cancer Res. 59:4004-4011 (1999)); rubromycin (Ueno, T. et al., Biochemistry 39:5995-6002 (2000)); diazaphilonic acid (Tabata, Y. et al., J. Antibiot. 52:412-414 (1999)); 9-Hydroxyellipticine (Sato, N. et al., FEBS Lett. 441:318-321 (1998)); and others known in the art.

In another embodiment, the small molecules comprise reporter compounds, particularly fluorescent, phosphorescent, radioactive labels, and detectable ligands. Useful fluorescent compounds include, by way of example and not limitation, fluorescein, rhodamine, TRITC, coumadin, Cy5, ethidium bromide, DAPI, and the like. Suitable fluorescent compounds are described in Haughland, R. P., Handbook of Fluorescent Probes and Research Chemicals Eugene, 9^{th} Ed., Molecular Probes, Oreg. (2003); incorporated herein by reference). Processing of the compositions by cell specific proteases releases inhibition and permits the cleaved composition to enter the cell and deliver the reporter compound into the target cell. Presence of a nuclear localization signal allows accumulation of the reporter compound within the cell. As further described in detail below, this property is useful in ascertaining the types of proteases expressed in a population of cells, and as a diagnostic method to identify or detect diseased cells.

Radioactive compounds are generally complexed or coupled to a component of the composition delivered into the cell. The cell penetrating peptide, the nuclear localization signal, or the cargo can be modified to carry the radioactive molecule. Radioactive compounds are useful as signals (e.g., tracers) or used to provide a therapeutic effect by specific delivery to a cell targeted (e.g., in the form of radiopharmaceutical preparations). Radioactive nuclides include, by way of example and not limitation, ³H, ¹⁴C, ³²P, ³⁵S, ⁵¹Cr, ⁵⁷Co ⁵⁹Fe, ⁶⁷Ga, ⁸²Rb, ⁸⁹Sr, ⁹⁹Tc, ¹¹¹In, ¹²³I, ¹²⁵I, ¹²⁹I, ¹³¹I, and ¹⁸⁶Re.

In yet a further embodiment, the small organic molecules are chelating ligands, or macrocyclic organic chelating molecules, particularly metal chelating compounds used to image intracellular ion concentrations or used as contrast agents for for medical imaging purposes. Chelating ligands are ligand that can bind with more than one donor atom to the same central metal ion. Chelators or their complexes have found applications as MRI contrast agents, radiopharmaceutical applications, and luminescent probes. Conjugates of chelating compounds useful for assessing intracellular ion concentrations may be voltage sensitive dyes and non-voltage sensitive dyes. Exemplary dye molecules for measuring intracellular ion levels include, by way of example and not limitation, Quin-2; Fluo-3; Fura-Red; Calcium Green; Calcium Orange 550 580; Calcium Crimson; Rhod-2 550 575; SPQ; SPA; MQAE; Fura-2; Mag-Fura-2; Mag-Fura-5; Di-4-ANEPPS; Di-8-ANEPPS; BCECF; SNAFL-1; SBFI; and SBFI.

In another embodiment, the ligands are chelating ligands that bind paramagnetic, superparamagnetic or ferromagnetic metals. These are useful as contrast agents for medical imaging and for delivery of radioactive metals to selected cells. Metal chelating ligands, include, by way of example and not limitation, diethylenetriaminepenta acetic acid (DTPA); diethylenetriaminepenta acetic acid bis(methylamide); macrocyclic tetraamine 1,4,7,10-tetraazacyclododecane-N,N′,N″, N′″-tetraacetic acid (DOTA); and porphyrins (see, e.g., The Chemistry of Contrast Agents in Medical Magnetic Resonance Imaging, Merbach A. E. and Toth E., Ed., Wiley Interscience (2001)). Paramagnetic metal ions, which are detectable in their chelated form by magnetic resonance imaging, include, for example, iron(III), gadolinium(III), manganese(II and III), chromium(III), copper(II), dysprosium(III), terbium(III), holmium(III), erbium(III), and europium(III). Paramagnetic metal ions particularly useful as magnetic resonance imaging contrast agents comprise iron(III) and gadolinium(III) metal complexes. Other paramagnetic, superparamagnetic or ferromagnetic are well known to those skilled in the art.

In another embodiment, the metal-chelate comprises a radioactive metal. Radioactive metals may be used for diagnosis or therapy based on delivery of small doses of radiation to a specific site in the body. Targeted metalloradiopharmaceuticals are constructed by attaching the radioactive metal ion to a metal chelating ligand, such as those used for magnetic imaging, and targeted delivery of the chelate complex to cells. An exemplary radioactive metal chelate complex is DTPA (see, e.g., U.S. Pat. No. 6,010,679).

Nucleic Acids

In one aspect, the compounds of interest comprise nucleic acids, including oligonucleotides and polynucleotides. By “nucleic acid” or “oligonucleotide” or “polynucleotide refers to at least two nucleotides covalently linked together. A nucleic acid of the present invention will generally contain phosphodiester bonds. However, in some cases, nucleic acid analogs are included that may have alternate backbones, comprising, for example, phosphoramide (Beaucage, S. L. et al., Tetrahedron 49:1925-63 (1993); Letsinger, R. L. et al., J. Org. Chem. 35: 3800-03 (1970); Sprinzl, M. et al., Eur. J. Biochem. 81:579-89 (1977); Letsinger, R. L. et al., Nucleic Acids Res. 14:3487-99 (1986); Sawai et al., Chem. Lett. 805 (1984); Letsinger, R. L. et al., J. Am. Chem. Soc. 110: 4470 (1988)), phosphorothioate (Mag, M. et al., Nucleic Acids Res. 19: 1437-41 (1991); and U.S. Pat. No. 5,644,048), phosphorodithioate (Briu et al., J. Am. Chem. Soc. 111: 2321 (1989)), O-methylphophoroamidite linkages (see, e.g., Eckstein, Oligonucleotides and Analogues: A Practical Approach, Oxford University Press (1991)), and peptide nucleic acid backbones and linkages (Egholm, M., Am. Chem. Soc. 114: 1895-97 (1992); Meier et al., Chem. Int. Ed. Engl. 31:1008 (1992); Egholm, M., Nature 365: 566-68 (1993); Carlsson, C. et al., Nature 380: 207 (1996)). Other analog nucleic acids include those with positive backbones (Dempcy, R. O. et al., Proc. Natl. Acad. Sci. USA 92: 6097-101 (1995)); non-ionic backbones (U.S. Pat. Nos. 5,386,023, 5,637,684, 5,602,240, 5,216,141 and 4,469,863; Kiedrowshi et al., Angew. Chem. Intl. Ed. English 30: 423 (1991); Letsinger, R. L. et al., J. Am. Chem. Soc. 110: 4470 (1988); and Letsinger, R. L. et al., Nucleoside & Nucleotide 13: 1597 (1994)). All publications are hereby expressly incorporated by reference.

The nucleic acids may be single stranded or double stranded, or contain portions of both double stranded or single stranded sequence. The nucleic acid may be DNA, both genomic and cDNA, RNA or hybrid, where the nucleic acid contains any combination of deoxyribo- and ribonucleotides, and any combination of bases, including uracil, adenine, thymine, cytosine, guanine, and any of known base analogs, including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5 carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracils, 5-methoxyaminomethyl-2-thiouracil, beta-D-maninosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

In one aspect the nucleic acids comprise functional nucleic acids. By “functional nucleic acid” refers to any nucleic acid that is bioactive. A functional nucleic acid may have enzymatic function, regulate transcription of nucleic acids, regulate the translation of an mRNA so as to interfere with the expression encoded protein, or affect other physiological processes in the cell. Functional nucleic acids include, by way of example and not limitation, ribozymes, antisense nucleic acids, decoy oligonucleotide nucleic acids, and interfering RNAs (RNAi).

In one embodiment, the nucleic acids comprise anti-sense nucleic acids. As used herein, “anti-sense nucleic acids” comprise nucleic acids, particularly in the form of oligonucleotides, characterized as hybridizing to the corresponding complementary or substantially complementary nucleic acid strand to inhibit expression of the gene encoded by the complementary strand.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. Generally, short oligonucleotides, of from 7 to 8 bases in length, can be strong and selective inhibitors of gene expression (see, e.g., Wagner et al., Nature Biotechnol. 14:840-844 (1996)).

Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation. The antisense nucleic acids may be directed to any expressed protein, including, by way of example and not limitation, to transcription factors, kinases, phosphorylases, telomerases, receptors, etc.

In one embodiment, the antisense nucleic acids are directed against telomerase (Norton, J. C. et al., Nat. Biotechonol. 14, 615-619 (1969); Pitts, A. E. and Corey, D. R., Proc. Natl. Acad. Sci. U.S.A. 95, 11549-11554 (1998); Elayadi, A. N. et al., Nucleic Acids Res. 29, 1683-1689 (2001); Tao, M. et al., FEBS Lett. 454, 312-316 ((1999)). In another embodiment, the antisense oligonucleotides are directed against receptors and components of cell signaling pathways. Various antisense oligonucleotides have been developed against cell signaling components. Exemplary antisense nucleic acids, include, by way of example and not limitation, the antisense nucleic acid directed against Raf-1 (Mullen, P. et al., Clin Cancer Res. 10(6):2100-2108 (2004), vascular endothelial zinc finger 1 (Vezf1), a zinc finger transcription factor expressed in endothelial cells (ECs) during vascular development (Miyashita, H. et al., Arterioscler Thromb Vasc Biol. epublication, March 18 (2004)); phosphorothioate antisense oligonucleotides to beta-catenin (Veeramachaneni, N. K. et al., J Thorac Cardiovasc Surg. 127(1):92-8 (2004)); and antisense oligonucleotides to Stat5 transcription factors (Xi, S. et al., Cancer Res. 63(20):6763-71 (2003)). It is to be understood that other antisense nucleic acids may be delivered into cells by the compositions described herein.

In another aspect, the nucleic acids are decoy oligonucleotids (ODN). The basis of the ODN decoy approach involves introducing into the cell a competing synthetic, transcription factor-specific consensus sequences or sequences that interact with other nucleic acid binding proteins. These synthetic decoys “compete” for binding of the protein (e.g., transcription factor) with consensus sequences in target genes. If delivered into the cell in sufficient concentrations these “decoys” have the potential to attenuate the binding of the nucleic acid binding protein, for example binding of transcription factors to promoter regions of target genes and thus attenuate the function of the protein to regulate the expression of its target gene(s). Generally, the decoy nucleic acids will comprises a minimal sequence bound by the nucleic acid binding protein. Transfected at high concentrations these decoys are shown to block activities of the nucleic acid binding proteins (see, e.g., Mann, M. J. and Dzau, V. J., J Clin Invest 106(9):1071-5 (2000)).

Thus in one embodiment, the sequences of the decoy nucleic acids are the sequences bound by a transcription factor. Exemplary ODN nucleic acid sequences have been described for transcription factors, including, by way of example and not limitation, NF-kB (nuclear factor-kappaB) (Sharma, H. W. et al., Anticancer Res. 16(1):61-9 (1996)); transcription factor E2F (Morishita, R. et al., Proc Natl Acad Sci USA 92(13):5855-9 (1995)); negative regulatory element (NRE) for the renin gene; angiotensinogen gene-activating element (AGE) for the angiotensinogen gene; and for the TERT Site C repressor protein, which inhibits expression of telomerase (U.S. Pat. No. 6,686,159; Morishita et al., Circ. Res. 82 (10):1023-8 (1998)).

In another embodiment, the decoy nucleic acid comprises a sequence bound by a viral protein involved in viral gene expression and replication. Exemplary nucleic acids for modulating viral acitivity included HIV TAR sequence, which regulates tat; HIV RRE sequence, which regulates rev to inhibit replication of the HIV virus (Sullenger, B. A. et al., Cell 63(3):601-8 (1990); Lee, S. W. et al., J. Virol. 68 (12): 8254-8264 (1994)); and ICP4 of herpes simplex virus type 1 required for viral replication (Clusel, C. et al., Gene Expr. 4(6):301-9 (1995)).

In another aspect, the nucleic acids for delivery into a taraget cell using the compositions of the present invention are interfering RNAs. RNAi, interfering RNA, or dsRNA mediated interference refers to double stranded RNAs capable of inducing RNA interference or RNA silencing (Bosher, J. M. et al., Nat. Cell Biol. 2: E31-36 (2000)). Introducing double stranded RNA can trigger specific degradation of homologous RNA sequences, generally within the region of identity of the dsRNA (Zamore, P. D. et. al., Cell 101: 25-33 (1997)). This provides a basis for silencing expression of genes, thus permitting a method for altering the phenotype of cells. The dsRNA may comprise synthetic RNA made by known chemical synthetic methods or by in vitro transcription of nucleic acid templates carrying promoters (e.g., T7 or SP6 promoters). The double stranded regions of the RNAi molecule are generally about 10-500 basepairs or more, preferably 15 basepairs, and most preferably 20-100 basepairs (see, e.g., Elbashir, S. M. et al., Genes Dev. 15(2): 188-200 (2001)).

RNAi sequences have been described for silencing gene expression in numerous organisms from plants, nematodes, trypanosomes, insects, and mammals. Exemplary RNAi sequences are described for cell surface receptor proteins integrins α3 and β1 (Billy, E. et al., Proc Natl Acad Sci USA 98(25):14428-33 (2001)); lamin B1, lamin B2, NUP153, GAS41, ARC21, cytoplasmic dynein, the protein kinase cdk1 and β- and γ-actin (Harborth, J. et al., J Cell Sci. 114:4557-65 (2001)); DNMT-1, which plays an role in CpG methylation and control of gene expression (Sui, G. et al., Proc Natl Acad Sci USA 99(8):5515-20 (2002)); β-arrestin (Sun, Y. et al., J. Biol. Chem. 277(51):49212-9 (2002); checkpoint kinase Chk-1 involved in regulating cell cycle progression in response to double-strand DNA breaks (Zhao, H. et al., Proc Natl Acad Sci USA 99(23):14795-800 (2002); hepatitis C virus replication using HCV specific RNAi sequences (Kapadia, S. B. et al., Proc Natl Acad Sci USA 100(4):2014-8 (2003)); homeobox transcription factor Rax involved in rat retina development (Matsuda, T. and Cepko, C. L., Proc Natl Acad Sci USA 101(1):16-22 (2004)); and ubiquitin conjugating enzyme (Habelhah, H. et al., EMBO J. 23(2):322-32 (2004)).

In yet a further embodiment, the compositions are used to deliver ribozymes or DNAzymes. Ribozymes and DNAzymes are nucleic acids capable of catalyzing cleavage of target nucleic acids in a sequence specific manner. Ribozymes include, among others, hammerhead ribozymes, hairpin ribozymes, and hepatitis delta virus ribozymes (Tuschl, T., Curr. Opin. Struct. Biol. 5:296-302 (1995)); Usman N., Curr. Opin. Struct. Biol. 6: 527-33 (1996)); Chowrira B. M. et al., Biochemistry 30: 8518-22 (1991)); Perrotta A. T. et al., Biochemistry 3:16-21 (1992)). As with antisense nucleic acids, nucleic acids catalyzing cleavage of target nucleic acids may be directed to a variety of expressed nucleic acids, including those of pathogenic organisms or cellular genes (see, e.g., Jackson, W. H. et al., Biochem. Biophys. Res. Commun. 245:81-84 (1998)). Catalytic DNA, or DNAzymes are RNA-cleaving DNA, which may offer a higher catalytic efficiency, specificity, and inherently greater stability than a typical ribozyme (Sun, L. Q. et al., Pharmacol. Rev. 52, 325-347 (2000)). Exemplary cell penetrating peptides conjugated to ribozyme directed against telomerase is described in Villa, R. et al., FEBS Letters 473(2):241-248 (2000);

It is to be understood that the person of ordinary skill in the art with the guidance provided herein can use the compositions to deliver nucleic acids other than those described above. For example, the nucleic acids may be candidate nucleic acids for use in screens for bioactive nucleic acid sequences.

Proteins and Peptides

In another embodiment, the compounds of interest comprise proteins. As used herein, a protein includes oligopeptides, peptides, and polypeptides. By “protein” herein is meant at least two covalently attached amino acids, which may be naturally occurring amino acids or synthetic peptidomimetic structures. The protein or peptide may be composed of naturally occurring and synthetic amino acids, including amino acids of (R) or (S) stereo configuration. Proteins including non-naturally occurring amino acids may be synthesized or in some cases, made by recombinant techniques (van Hest, J. C. et al., FEBS Lett. 428: 68-70 (1998); and Tang et al., Abstr. Pap. Am. Chem. S218: U138-U138 Part 2 (1999)), both of which are expressly incorporated by reference herein).

In one aspect, the compounds of interest are peptide tags used for purposes of detection, particularly through the use of antibodies directed against the peptide. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Mol. Cell. Biol. 5:3610-3616 (1985)); and the Herpes Simplex virus glycoprotein D (gD) tag and its antibody (Paborsky et al., Protein Engineering 3:547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Nat. Acad. Sci. USA 87:6393-6397 (1990)).

In another aspect, the proteins or peptides comprise detectable enzymes or other reporter proteins. Enzymes and reporter proteins include, by way of example and not limitation, green fluorescent protein (Chalfie, M. et al., Science 263: 802-05 (1994)); Enhanced GFP (Clontech; Genbank Accession Number U55762); blue fluorescent protein (BFP, Quantum Biotechnologies, Inc. 1801 de Maisonneuve Blvd. West, 8th Floor, Montreal (Quebec) Canada H3H 1J9; Stauber, R. H., Biotechniques 24: 462-71 (1998); Heim, R. et al., Curr. Biol. 6: 178-82 (1996)), enhanced yellow fluorescent protein (EYFP, Clontech Laboratories, Inc., 1020 East Meadow Circle, Palo Alto, Calif. 94303), Anemonia majano fluorescent protein (amFP486, Matz, M. V., Nat. Biotech. 17: 969-73 ((1999)), Zoanthus fluorescent proteins (zFP506 and zFP538; Matz, supra), Discosoma fluorescent protein (dsFP483, drFP583; Matz, supra), Clavularia fluorescent protein (cFP484; Matz, supra); luciferase (for example, firefly luciferase, Kennedy, H. J. et al., J. Biol. Chem. 274: 13281-91 (1999); Renilla reniformis luciferase (Lorenz, W. W., J Biolumin. Chemilumin. 11: 31-37 (1996)); Renilla muelleri luciferase (U.S. Pat. No. 6,232,107); β-galactosidase (Nolan, G. et al., Proc. Natl. Acad. Sci. USA 85: 2603-07 (1988)); β-glucouronidase (Jefferson, R. A. et al. EMBO J. 6: 3901-07 (1987); Gallager, S., GUS Protocols: Using the GUS Gene as a reporter of gene expression, Academic Press, Inc. (1992)); and alkaline phosphatase (Cullen, B. R. et al., Methods Enzymol. 216: 362-68 (1992)).

In another embodiment, the proteins and peptide may comprise toxins that cause cell death, or impair cell survival when introduced into a cell. A suitable toxin is campylobacter toxin CDT (Lara-Tejero, M., Science 290:354-57 (2000)). Expression of the CdtB subunit, which has homology to nucleases, causes cell cycle arrest and ultimately cell death. Another exemplary toxin is diptheria toxin (and similar Pseudomonas exotoxin), which functions by ADP ribosylating ef-2 (elongation factor 2) molecule in the cell and preventing translation. Expression of the diptheria toxin A subunit induces cell death in cells expressing the toxin fragment. Other useful toxins include cholera toxin and pertussis toxin (catalytic subunit-A ADP ribosylates the G protein regulating adenylate cyclase), pierisin from cabbage butterflys, an inducers of apoptosis in mammalian cells (Watanabe, M., Proc. Natl. Acad. Sci. USA 96:10608-13 (1999)), phospholipase snake venom toxins (Diaz, C. et al., Arch. Biochem. Biophys. 391:56-64 (2001)), ribosome inactivating toxins (e.g., ricin A chain, Gluck, A. et al., J. Mol. Biol. 226:411-24 (1992)); and nigrin (Munoz, R. et al., Cancer Lett. 167: 163-69 ((2001)).

In yet a further embodiment, the proteins or peptides to be delivered are protein domains, or peptide mimicks thereof, that interact with other biological molecules. A protein-interaction domain refers to a protein region or sequence that interacts with other biomolecules, including other proteins, nucleic acids, lipids, etc. These protein domains frequently act to provide regions that induce formation of specific multiprotein complexes for recruiting and confining proteins to appropriate cellular locations or affect specificity of interaction with target ligands. Protein-interaction domains comprise modules or micro-domains ranging about 20-150 amino acids that can be expressed in isolation and bind to their physiological partners. Many different interaction domains are known, most of which fall into classes related by sequence or ligand binding properties. Accordingly, the interaction domains may comprise proteins that are members of these classes of protein domains and their relevant binding partners. These include, among others, SH2 domains (src homology domain 2), SH3 domain (src homology domain 3), PTB domain (phosphotyrosine binding domain), FHA domain (forkedhead associated domain), WW domain, 14-3-3 domain, pleckstrin homology domain, C1 domain, C2 domain, FYVE domain (Fab-1, YGL023, Vps27, and EEA1), death domain, death effector domain, caspase recruitment domain, Bcl-2 homology domain, bromo domain, chromatin organization modifier domain, F box domain, hect domain, ring domain (Zn+2 finger binding domain), PDZ domain (PSD-95, discs large, and zona occludens domain), sterile a motif domain, ankyrin domain, arm domain (armadillo repeat motif), WD 40 domain and EF-hand (calretinin), PUB domain (Suzuki T. et al., Biochem. Biophys. Res. Commun. 287: 1083-87 (2001)), nucleotide binding domain, Y Box binding domain, H.G. domain, all of which are well known in the art. Since protein interaction domains are pervasive in cellular regulation, such as signal transduction cascades and transcription factors, introduction of protein or peptide interaction domains acting in a specific regulatory pathway may provide a basis for inactivating or activating such pathways in normal and diseased cells.

It is to be understood that other peptide compounds besides transcription factor modulators may be delivered to the nucleus to target other nuclear acting components. Thus, for example it is known that telomerase activity may be inhibited by overexpression of PinX1 or its 100 amino acid carboxy fragment (Zhou, X. Z. and Lu, K. P.; Cell 107(3) 347-359 (2001)). Expression of these peptides have been shown to inhibit tumorigenesis in mice.

Synthesis of Compositions

Chemical Synthesis

Synthesis of the compositions described herein may use any chemical synthetic techniques known in the art for the preparation of the peptides and peptide analogs. In one aspect, the compositions may be prepared using conventional solution or solid phase peptide synthesis and standard chemistries. Use of amino acid analogues derivatized for use in standard synthesis chemistries, including D-isomer amino acids, or modifications following peptide synthesis may be used to generate peptide analogues. General synthetic methods are described in “Solid Phase Peptide Synthesis” in Methods in Enzymology (Fields, G. B. Ed.) Academic Press, San Diego (1997)); Lloyd-Williams, P. et al., Chemical Approaches to the Synthesis of Peptides and Proteins CRC Press, Boca Raton. (1997)). Other references describing synthesis of peptides and peptide analogues include, among others, Wipf, P. and Henninger, T. C., J. Org. Chem. 62:1586-1587 (1997); Wellings, D. A. and Atherton, E., “Standard Fmoc protocols,” in Methods Enzymol. 289, 44-67 (1997) Walker, M. A., Angew. Chem. Int. Ed. 36, .1069-1071 (1997); Suhara, Y. et al., Tetrahedron Lett. 38:7167-7170 (1997); Songster, M. F. and Barany, G., “Handles for solid-phase peptide synthesis,” in Methods Enzymol. 289, 126-174 (1997); Scott, W. L. et al., Tetrahedron Lett. 38, 3695-3698 (1997); O'Donnell, M. J. et al., Tetrahedron Lett. 38:7163-7166 (1997); Muir, T. W. et al., “Protein synthesis by chemical ligation of unprotected peptides in aqueous solution,” in Methods Enzymol. 289:266-298 (1997); Royo, M. et al., Eur. J. Org. Chem. 45-48 (2001)); and Stewart, J. M., “Cleavage methods following Boc-based solid-phase peptide synthesis,” Methods Enzymol. 289:29-44 (1997)).

As will be appreciated by those skilled in the art, segment condensation may be used to synthesize the compositions (Kimura, T. et al., Biopolymers 20:1823-1832 (1981); Sakakibara, S., Biopolymers 37:17-28 (1995); and Canne, L. E. et al., J. Am. Chem. Soc. 121:8720-8727 (1999)). In segment condensation, peptide segments of the final peptide product are synthesized separately and then assembled to form the full length peptide product (see, e.g., Nishuchi, Y. et al., Proc. Natl. Acad. Sci. USA 95:13549-13554 (1998)). Depending on the synthetic strategy, solution or solid phase based ligation of the peptide segments may be used.

Disulfide linkages, if desired, may be formed after peptide synthesis. Formation of the disulfide linkages is performed in the presence of mild oxidizing agents. Chemical oxidizing agents or exposure to oxygen may be used to effect the linkages. Methods known in the art include those described in Stewart et al., Solid Phase Peptide Synthesis, 2nd Ed., Pierce Chemical Company, Rockford, Ill. (1984); and Ahmed et al., J. Biol. Chem. 250:8477-8482 (1975). A method for generating disulfide linkages on solid support is described in Albericio, Int J. Peptide Protein Res. 26:92-97 (1985).

The terminal amino group or carboxyl group of the oligopeptide may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18, carbon atoms. The peptide or derivatives thereof may also be modified by acetylation or methylation to alter the chemical properties, for example lipophilicity. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see, e.g., Creighton, T. E., Proteins: Structure and Molecular Properties, W.H. Freeman & Co. San Francisco, Calif. (1983)).

Generally, various elements of the compositions are ordered in such a way as to preserve the various biological features of the compositions. Thus, the components of the compositions are operably linked into a functional relationship with other components of the compositions. For example, the inhibitor of cell pepenetrating peptide activity is operably linked to the cleavage site and the cell penetrating peptide if it inhibits cell penetrating peptide activity but does not inhibit upon cleavage at the protease recognition site. If all of the components including the therapeutic agents are peptides, the composition may be operably linked to other components by synthesizing the composition as a contiguous peptide or protein. In other embodiments, the therapeutic agent may be coupled to the peptide portions via non-peptide linkers/crosslinking agents, as further described below.

In one embodiment, the inhibitor of cell penetrating peptide is adjacent to the cell penetrating activity, preferably attached or linked to the amino terminus of the cell penetrating peptide. The cargo or compound is linked or conjugated, directly or indirectly, to the cell penetrating peptide portion. A cleavage site is present in between the inhibitor portion and the cell penetrating portion such that cleavage results in separation of the inhibitor away from the translocating peptide. A subcellular localization sequence, if present, is placed in such a manner as to maintain the linkage to the cell penetrating peptide and cargo upon cleavage of the composition. Thus, for example, a nuclear localization signal may be added to the carboxy terminus of the cell penetrating peptide while the cargo or compound is attached to the nuclear localization signal. Upon cleavage and removal of inhibitor, a modified composition comprising the cell penetrating peptide, a subcellular localization signal, and the cargo is a single complex that enters the cell.

An illustration of one arrangement of the composition is as follows:
ICPP-CS-CPP-NLS-COI
where ICPP is the inhibitor of cell penetrating peptide, CS is the cleavage site, CPP is the cell penetrating peptide, NLS is the nuclear localization sequence, and COI is the compound of interest. When peptides with multiple activities, such as cell penetrating peptide merged to a cleavage site, are used, the following arrangements may be contemplated:
ICPP/CS-CPP/NLS-COI
where ICPP/CS is a peptide with cell penetrating peptide merged with a cleavage site, CPP/NLS is a peptide with cell penetrating peptide merged with a nuclear localization signal, and COI is the compound of interest. It is to be understood that the compositions of the invention are not limited to the constructions described above, and that other constructs may be made having the desired biological characteristics.

To maintain activity of the various portions or elements of the compositions, linkers may be used. The linkers may be chemical linkers, nucleic acid linkers, or peptide linkers, as is well known in the art and as described herein. Peptide linkers are useful when the inhibitor of cell penetrating peptide, the cell penetrating peptide, and subcellular localizations signal are made as a single contiguous peptide or protein. Useful linkers include glycine polymers (G)ₙ, glycine-serine polymers (including, for example, (GS)ₙ, (GSGGS)ₙ and (GGGS)ₙ where n is an integer of at least one), glycine-alanine polymers, alanine-serine polymers, and other flexible linkers as will be known and appreciated by those in the art. Glycine and glycine-serine polymers are advantageous since both of these amino acids are relatively unstructured, and therefore may be able to serve as a neutral tether between components. Thus, linkers may be used to link the cell penetrating peptide to the subcellular localization signal as well as for attaching the cargo.

Recombinant Synthesis

In another aspect, the compositions are synthesized using recombinant nucleic acids made by conventional recombinant genetic engineering techniques. As used herein, “recombinant nucleic acid” refers to a nucleic acid initially formed in vitro, generally by the manipulation of the nucleic acid by polymerases, endonucleases, and ligases, in a form not found in nature. For example, an isolated nucleic acid or an expression formed in vitro by ligating nucleic acid molecules that are not normally joined, are considered recombinant molecules. It is to be understood that a recombinant nucleic acid introduced into a suitable host cell or organism may replicate, generally by using the in vivo cellular machinery of the host cells rather than the in vitro manipulations. Such nucleic acids, although replicated non-recombinantly are still considered recombinant for the purposes of the invention. The compositions described herein may be produced recombinantly using nucleic acids capable of expressing the peptides.

For recombinant production, a polynucleotide sequence encoding the peptide is made and inserted into an appropriate expression vehicle, i.e., a vector which contains the necessary elements for transcription and translation of the inserted coding sequence. The recombinant construct is generally made by operably linking nucleic acid segments encoding the various components of the compositions to form a fusion nucleic acid capable of expressing the composition having the desired biological characteristics. Typical arrangements of the nucleic acid segments will be made based on relationships of the components described above (see section 5.5.1). The expression vehicle is then introduced into a suitable host or target cell which is capable of expressing the peptide. The expressed product, i.e., the recombinant peptide/protein may be isolated by well established procedures. General descriptions of recombinant techniques, including expression of recombinant peptides products are provided in, among others, Sambrook, J. et al., Molecular Cloning: A Laboratory Manual, 3^{rd} Ed., Cold Spring Harbor Laboratory, N.Y. (2001); and Ausubel, F. et al., Current Protocols in Molecular Biology, updates to 2004, Greene Publishing Associates and Wiley Interscience, N.Y. (2004).

To increase efficiency of production, the nucleic acids can be designed to encode multiple units of the peptides, either as homopolymers or heteropolymers, where each unit peptide is separated by a chemical or enzymatic cleavage site. The polypeptide produced from the nucleic aicds can be cleaved to generate the peptide units of the compositions. In another embodiment, a polycistronic message can be made such that a single mRNA species encodes multiple peptides. Each coding region is operably linked to a internal ribosome entry site (IRES). Because each IRES element initiates translation of each peptide linked to the IRES sequence, translation of multiple, individual peptides can take place.

In another embodiment, nucleic acids comprise sequences containing codons replaced with degenerate codons coding for the same amino acid. This arises from the degeneracy of the genetic code where the same amino acids are encoded by alternative codons. Replacing one codon with another degenerate codon changes the nucleotide sequence without changing the amino acid residue. An extremely large number of nucleic acids may be made, all of which encode the compositions of the present invention. In this regard, the present invention has specifically contemplated each and every possible variation of polynucleotides that could be made by selecting combinations based on the possible codon choices, and all such variations are to be considered specifically disclosed.

Changing the codons may be desirable for a variety of situations. For example, substitutions with a degenerate codon is useful when eliminating cryptic splice signals present in the coding regions of the nucleic acid, creating alternative primers for amplification reactions, and particularly for changing the expression levels of the encoded protein. Thus contemplated in the present invention are codon optimized nucleic acids for expression in a particular organism. By “codon optimized” herein is meant changes in the codons to those preferentially used in a particular organism such that the gene is efficiently expressed in the organism. By “preferred”, “optimal” or “favored” codons, or “high codon usage bias” or grammatical equivalents as used herein is meant codons used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof.

A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see, e.g., GCG CodonPreference, Genetics Computer Group Wisconsin Package; CodonW, John Peden, University of Nottingham; McInerney, J. O., Bioinformatics 14: 372-373 (1998); Stenico, M. et al., Nucleic Acids Res. 22:2437-2446 (1994); Wright, F., Gene 87: 23-29 (1990)). Codon usage tables are available for a growing list of organisms (see, e.g., Wada, K. et al., Nucleic Acids Res. 20:2111-2118 (1992); Nakamura, Y. et al., Nucleic Acids Res. 28:292 (2000)).

Various host-expression vector systems may be used to express the peptide compositions described herein. These include, but are not limited to, microorganisms such a bacteria transformed with recombinant phage or plasmid expression vectors containing the appropriate coding sequence, yeast or filamentous fungi transformed with recombinant yeast or fungi expression vectors containing the appropriate coding sequence. Expression is done in a wide range of host cells that span prokaryotes and eukaryotes, including bacteria, yeast, plants, insects, and animals. The peptides may be expressed in, by of example and not limitation, E. coli, Saccharomyces cerevisiae, Saccharomyces pombe, Tobacco or Arabidopsis plants, insect Schneider cells, and mammalian cells, such as COS, CHO, HeLa, and the like, either intracellularly or in a secreted form by fusing the peptides to an appropriate signal peptide. Secretion from the host cell may be done by fusing the DNA encoding the composition and a DNA encoding a signal peptide. Secretory signals are well known in the art for bacteria, yeast, insect, plant, and mammalian systems.

Varieties of techniques are available for introducing proteins and nucleic acids into cells. By “introduced” into herein is meant that protein is delivered into the cell or that a nucleic acid enters the cells in a manner suitable for subsequent expression of the nucleic acid. Technique used for delivery into cells will vary depending on the nature of the composition and whether cells are in vitro, ex vivo, or in vivo, and the type of cell or host organism. When cells are treated ex vivo, the cells may be autologous cells, which are cells obtained from the animal prior to reintroduction into the same organism. Exemplary techniques for introducing proteins and nucleic acids into cells include the use of liposomes, Lipofectin®, electroporation (in vivo and in vitro), microinjection, cell fusion, DEAE dextran, calcium phosphate precipitation, viral vectors, and biolistic particle bombardment. Those skilled in the art can choose the method appropriate for the particular application.

Depending on the host and vector systems employed, the expression vectors are either self-replicating extrachromosomal vectors or vectors that integrate into the host chromosome, for example vectors based on retroviruses, vectors with site specific recombination sequences, or by homologous recombination. Generally, these vectors include control sequences operably linked to the nucleic acids encoding the oligopeptides. By “control sequences” is meant nucleic acid sequences necessary for expression of the subject peptides in a particular host organism. Thus, control sequences include sequences required for transcription and translation of the nucleic acids, including, but not limited to, promoter sequences, enhancer or transcriptional activator sequences, ribosomal binding sites, transcriptional start and stop sequences; polyadenylation signals; etc.

When cloning in bacterial systems, the expression vectors are bacterial expression vectors including, among others, vectors for Bacillus subtilis, E. coli, Haemophilus, Streptococcus cremoris, and Streptococcus lividans, and use any number of transcription and translation elements for expression in the host. For example, inducible promoters include inducible promoters from bacteriophage (e.g., pL), plac and ptrp, may be used. In addition, synthetic promoters and hybrid promoters are also useful; for example, the tac promoter, which is a hybrid of the trp and lac promoter sequences.

In another embodiment, the expression vectors are used to express the compositions in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL promoters (e.g., GAL 1, GAL 4, GAL 10 etc.), the promoters from alcohol dehydrogenase (ADH or ADC1), enolase, glucokinase, glucose 6-phosphate isomerase, glyceraldehyde-3-phosphate-dehydrogenase, hexokinase, phosphofructokinase, 3-phosphoglycerate mutase, pyruvate kinase, fructose bisphosphate, acid phosphatase gene, tryptophase synthase (TRP5) and copper inducible CUP1 promoter. Any plasmid containing a yeast compatible promoter, an origin of replication, and termination sequences is suitable.

In another preferred embodiment, the expression vectors are used for expression in plants. Vectors are known for expressing genes in Arabidopsis thaliana, tobacco, carrot, and maize and rice cells. Suitable promoters for use in plants include those of plant or viral origin, including, but not limited to CaMV 35S promoter (active in both monocots and dicots; Chapman, S. et al., Plant J. 2:549-557 (1992)) nopoline promoter, mannopine synthase promoter, soybean or Arabidopsis thaliana heat shock promoters, tobacco mosaic virus promoter (Takmatsu et al., EMBO J. 6: 307 (1987)), and AT2S promoters of Arabidopsis thaliana (i.e., PAT2S1, PATS2, PATS3 etc.). In another embodiment, the promoters are tissue specific promoters active in specific plant tissues or cell types (e.g., roots, leaves, shoot meristem, etc.), which are well known in the art. Alternatively, the expression vectors comprise recombinant plasmid expression vectors based on Ti plasmids or root inducing plasmids.

In yet another embodiment, the expression vectors are used to express the compositions in insects and insect cells. In one embodiment, fusion proteins are produced in insect cells. Expression vectors for the transformation of insect cells, and in particular, baculoviral vectors used to create recombinant baculoviruses for expressing foreign genes, are well known in the art (see, e.g., O'Reilly, D. R. et al., Baculovirus Expression Vectors: A Laboratory Manual, W.H. Freeman & Co, New York (1992)). By “baculovirus” or “nuclear polyhedrosis viruses” as used herein refers to expression systems using viruses classified under the family of baculoviridae, preferably subgroup A. In another embodiment, these include expression systems specific for Bombix, Autographica, and Spodoptera cells (see, e.g., U.S. Pat. No. 5,194,376). Other expression systems include Amsacta moorei entomopoxvirus (AmEPV), Aedes aegypti desonucleosis (Aedes DNV; U.S. Pat. No. 5,849,523), and Galleria mellonella densovirus (GmDNV; Tal et al., Arch. Insect Biochem. Physiol. 22:345-356 (1993)); Rong, Y. S., Science 288:2013-18 (2000)), site directed recombination (e.g., cre-lox), and transposon mediated integration (e.g., P-element transposition elements).

In a further embodiment, the compositions are expressed in mammalian cells. The mammalian vectors will generally include inducible and constitutive promoters; a transcription initiating region, generally located 5′ to the start of the coding region; and a TATA box, present at about 25-30 basepairs upstream of the transcription initiation site. The promoter will also contain upstream regulatory elements that control the rate and initiation of transcription, including CAAT and GC box, enhancer sequences, and repressor/silencer sequences (see, e.g., Chang B. D., Gene 183: 137-42 (1996)). These promoter controlling elements may act directionally, requiring placement upstream of the promoter region, or act non-directionally. These aforementioned transcriptional control sequences may be provided from non-viral or viral sources. Commonly used promoters and enhancers are from viral sources since the viral genes have a broad host range and produce high expression rates. Viral promoters, including upstream controlling sequences, may be from polyoma virus, adenovirus 2, simian virus 40 (early and late promoters), and herpes simplex virus (e.g., HSV thymidine kinase promoter), human cytomegalovirus promoter (CMV), and mouse mammary tumor virus (MMTV-LTR) promoter. A variety of non-viral promoters with constitutive, inducible, cell specific, or developmental stage specific activities are also well known in the art (e.g., β-globin promoter, mammalian heat shock promoter, metallothionein, ubiquitin C promoters, EF-1alpha promoters, etc.). Cell specific promoters include, among others, promoters active in olfactory bulb, thyroid, lung, muscle, pancreas, liver, lung, heart, breast, prostate, kidney, etc. Promoters and promoter controlling elements are chosen based on the desired level of promoter activity and the cell type in which the compositions of the present invention are to be expressed.

Additional sequences in the expression vectors include splice sites for proper expression, polyadenylation signals, 5′ CAP sequence, transcription termination sequences, and the like. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation. Examples of transcription terminator and polyadenylation signals include those derived from SV40.

Other expression systems for producing the compositions will be apparent to those of ordinary skill in the art.

Coupling or Linking of Cargo and Compounds

By “linked” as used herein is meant that the elements or portions of the compositions are associated with one another. Examples of such methods of linking include (1) when the compound of interest is a peptide, the peptides components or portions can be linked by a peptide bond, i.e., the peptides can be synthesized contiguously; (2) when the compound or cargo is a polypeptide or a protein, the cell penetrating peptide, or a nuclear localization peptide, if present, can be linked to the peptide cargo by a peptide bond or by a non-peptide covalent bond (such as conjugating with a crosslinking reagent); (3) for molecules that have a negative charge, such as nucleic acids, the molecule and the signal peptide (and a nuclear localization peptide, if desired) can be joined by charge-association between the negatively-charged molecule and the positively-charged amino acids in the peptide or by other types of association between nucleic acids and amino acids; (4) chemical ligation methods can be employed to create a covalent bond between the carboxy-terminal amino acid of the signal peptide (or a nuclear localization peptide, if desired) and the compound.

When the compositions are not expressed as a contiguous protein or peptide, the linking of compounds of interest to form the compositions with attached compounds may be made through functional groups on the compounds of interest. Typical functional groups include the amino terminal of the peptide, epsilon amino group of lysine, thiol groups on cystein, and carboxy terminus of the peptide. Various linkers and crosslinking agents suitable for conjugation or crosslinking are described in Hermanson, G. T., Bioconjugate Techniques, Academic Press, San Diego, Calif. (1996); Pierce: Applications Handbook & Catalog, Perbio Science, Ermbodegem, Belgium (2003-2004); Haughland, R. P., Handbook of Fluorescent Probes and Research Chemicals Eugene, 9^{th} Ed., Molecular Probes, OR (2003); and U.S. Pat. No. 5,747,641; all references incorporated herein by reference. Exemplary coupling or linking reagents include, by way of example and not limitation, hemi-succinate esters of N-hydroxysuccinimide; sulfo-N-hydroxy-succinimide; hydroxybenzotriazole, and p-nitrophenol; dicyclohexylcarbodiimide (DCC), 1-(3-dimethylaminopropyl)-3-ethylcarbodiimide (ECD), and 1-(3-dimethylaminopropyl)-3-ethylcarbodiimide methiodide (EDCI) (see, e.g., U.S. Pat. No. 4,526,714) the disclosure of which is fully incorporated by reference herein. Other linking reagents include glutathione, 3-(diethoxyphosphoryloxy)-1,2,3-benzotriazin-4(3H)-one (DEPBT), onium salt-based coupling reagents, polyoxyethylene-based heterobifunctional cross-linking reagents, and other reagents that facilitate the coupling of organic drugs and peptides to various ligands (Haitao, et al., Organ Lett 1:91-94 (1999); Albericio et al., J Organic Chemistry 63:9678-9683 (1998); Arpicco et al., Bioconjugate Chem. 8:327-337 (1997); Frisch et al., Bioconjugate Chem. 7:180-186 (1996); Deguchi et al., Bioconjugate Chem. 10:32-37 (1998); Beyer et al., J. Med. Chem. 41:2701-2708 (1998); Drouillat et al., J. Pharm. Sci. 87:25-30 (1998); Trimble et al., Bioconjugate Chem. 8:416-423 (1997)).

Salts of the Compositions

The compositions of the present invention can be used in the free acid/base form, in the form of pharmaceutically acceptable salts, or mixtures thereof, as is known in the art. Such salts can be formed, for example, with organic anions, organic cations, halides, alkaline metals, etc.

The term “pharmaceutically acceptable salts” embraces salts commonly used as salts and addition salts of free acids or free bases. The nature of the salt is not critical, provided that it is pharmaceutically acceptable. Suitable pharmaceutically acceptable base addition salts of the present compositions include metallic salts and organic salts.

Suitable inorganic salts may be chosen from appropriate alkali metal (group Ia) salts, alkaline earth metal (group IIa) salts, and other physiologically acceptable metals. Such salts can be prepared, for example, from aluminum, calcium, lithium, magnesium, potassium, sodium, and zinc.

Organic salts can be prepared from tertiary amines and quaternary ammonium salts, including in part, tromethamine, diethylamine, N,N′-dibenzyl-ethylenediamine, chloroprocaine, choline, diethanolamine, ethylenediamine, meglumine (N-methyl-glucamine), and procaine. Other organic salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

The basic nitrogen-containing groups can be quarterized with agents such as lower alkyl halides, such as methyl, ethyl, propyl, and butyl chloride, bromides, and iodides; dialkyl sulfates such as dimethyl, diethyl, dibuytl, and diamyl sulfates; long chain halides such as decyl, lauryl, myristyl, and stearyl chlorides, bromides, and iodides; aralkyl halides such as benzyl and phenethyl bromides; and others.

All of these salts can be prepared by conventional means from the corresponding compositions disclosed herein by reacting with the appropriate acid or base therewith. Water- or oil-soluble or dispersible products are thereby obtained as desired.

Purification of Compositions

The compositions can be purified by art-known techniques such as reverse phase chromatography, high performance reverse chromatography, ion exchange chromatography, gel electrophoreisis, affinity chromatography, molecular sieve chromatography, isoelectric focusing, and the like. In a preferred embodiment, the compositions of the present invention may be purified or isolated after synthesis or expression. By “purified” or “isolated” is meant free from the environment in which the composition is synthesized or expressed, and in a form where it can be practically used. In one aspect, purified or isolated is meant that the composition is substantially pure, i.e., more than 90% pure, preferably more than 95% pure, and preferably more than 99% pure. Compositions, particularly peptides, may also be purified by selective solubility, for instance in the presence of salts or organic solvents. The degree of purification necessary will vary depending on use of the subject compositions. Thus, in some instances no purification will be necessary.

For affinity purification, antibodies that specifically bind the compositions may be used. Polyclonal antibodies may be made by immunizing suitable host animals by inoculation with the compositions or portions of the compositions (e.g., cell penetrating peptide, peptide cargo, etc.). Host animals include, but are not limited to, rabbits, mice, guinea pigs, rats, goats, donkeys, horses, and the like. An adjuvant may be used to enhance the immune response. The compositions may also be conjugated to naturally occurring or synthetic peptides to provide a carrier immunogen for generating antibodies to the subject compositions. Suitable carriers for generating antibodies include, among others, hemocyanins (e.g., Keyhole Limpet hemocyanin—KLH); albumins (e.g., bovine serum albumin, ovalbumin, human serum albumin, etc.); immunoglobulins; thyroglobulins (e.g., bovine thyroglobulin); toxins (e.g., diptheria toxoid, tetanus toxoid); and polypeptides such as polylysine or polyalanine-lysine. Although proteins are preferred carriers, other carriers, preferably high molecular weight compounds, may be used, including carbohydrates, polysaccharides, lipopolysaccharides, nucleic acids, and the like of sufficient size and immunogenicity.

Monoclonal antibodies to the composition may be prepared by using any known technique for producing monoclonal antibodies. Monoclonal antibodies may be prepared using hybridoma methods, such as those described by Kohler and Milstein, Nature, 256:495 (1975); the human hybridoma technique as described by Kosbor et al., Immunology Today 4:72 (1983); and the EBV hybridoma technique. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567. DNA encoding the monoclonal antibodies against the compositions of the invention can be readily isolated and sequenced using conventional procedures (e.g., by using oligonucleotide probes that are capable of binding specifically to genes encoding the heavy and light chains of murine antibodies). The hybridoma cells producing the appropriate antibodies are a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody. Humanized forms of non-human (e.g., murine) antibodies may also be made and used (Jones et al., Nature 321:522-525 (1986); Riechmann et al., Nature 332:323-329 (1988); and Presta, Curr. Op. Struct. Biol. 2:593-596 (1992)).

Antibody may also comprise fragments of antibodies generated by known techniques. For example, fragments include F(ab)2 produced by pepsin digestion of the antibody molecule, and Fab fragments generated by reducing the disulfide linkages of the F(ab″)2 fragments. In another embodiment, Fab fragments may be constructed and screened for Fab fragments with the desired specificity for the compositions (Huse et al., Science 246:1275-1281 (1989)).

Affinity purification using the antibodies may be done by attaching it to a support, such as agarose or polyacrylamide, and the antibody-support used to purify the compositions (see, e.g., Livingstone, “Immunoaffinity Chromatography of Proteins,” in Methods in Enzymology 34:723-731 (1974)).

Methods of Use

In the present invention, delivery of compounds of interest or a cargo into a cell with the compositions may be used in a variety of formats. Generally, these include assays to identify proteases specifically expressed or upregulated in certain cells or tissues, where the proteases are useful as markers for cell development, including disease development, and as reporters of biological processes within the cell. In another embodiment, the compositions are used to deliver bioactive compounds into cells, in particular the delivery into the cell nucleus of activators or inhibitors of nuclear acting factors, for determining their function within cells or as a therapeutic treatment for a condition or disease. These and other uses are comtemplated for the compositions of the present invention.

Assay for Cell Specific Proteases

In one aspect, the compositions of the present invention are used to assay for proteases upregulated or expressed in specific tissues and/or cell types. In this embodiment, cells are contacted with different compositions, where compositions have different protease substrate sequences. A reporter molecule whose signal (i.e., spectral signature) is uniquely associated with a specific cleavage sequence is attached to the compositions. Protease mediated cleavage of the substrates will lead to entry of the cleaved composition into the cell via membrane translocating activity of the cell penetrating peptide. Delivery of the reporter molecule into the cell and subsequent detection of the unique reporter molecule provides information on the type of protease produced by the cell type. Using this information, the appropriate cell delivery composition may be used to deliver therapeutic compounds into the cells, as further described below.

Furthermore, the protease activity profile determined for various cell types, including normal and/or disease affected cells, may be used as markers for determining the type of disease or disease severity. For example, in estrogen-receptor-positive human breast cancer cell lines (MCF7, ZR75-1), estrogen stimulates the secretion of a 52,000 Da (52K) glycoprotein protease into the culture medium, which stimulates cell proliferation. In another example, clinical studies using both immunohistochemistry and immunoenzymatic assay of breast cancer cytosol have shown that the concentration of total cellular cathepsin D correlates with the proliferation of mammary ducts and is also a useful prognostic indicator of breast cancer (Rochefort H, Biochimie. 70(7):943-949 (1988). Other proteases suggested a indicators of disease conditions include plasminogen activators (PAs), which emerge in late stages of cutaneous melanocytic tumour progression, and cathepsin B, which activity is increased in most malignant tumors and is associated with tumor progression (Berquin, I. M. et al., Adv Exp Med Biol. 389:281-94 (1996)).

The compositions of the present invention are used in the contexts described above to determine the presence of proteases in the various conditions. The cargo or compounds are reporter molecules, such as fluorescent compounds. A plurality of compositions, where each compostion comprises a different cleavage site, is contacted with the cells to be tested. By coupling distinguishable fluorescent compounds, entry of specific fluors can be correlated to expression of certain proteases, and consequently provide a diagnostic marker for protease activity.

Medical Imaging

In yet a further aspect, the compositions are used in medical imaging procedures. In one embodiment, the cargoes may be metal chelate complexes delivered to cells. As provided above, an example of metal chelate complexes used in medical imaging is DPTA complexed to gadolinium(III). Alternatively, a radioactive metal can be used for detection purposes. The protease cleavage sites comprise sequences recognized by the proteases expressed by the target cells, particularly metatstatic tumors. Following administration of the compositions, proteases expressed by the tumor cells cleave the compositions, thereby releasing inhibition of cell penetrating peptide and permitting tranduction of the metal-chelate complex into the tumor cell or cells located proximately to the tumor site.

Imaging of the paragmagnetic metal by magnetic resonance imaging (MRI), or detection of the radioactive compound by positron emission tomography (PET), should permit detection of the tumor mass. This can be extended to other diseases where presence of extracellular protease is a marker for the disease condition.

Treatment

In another aspect, the compositions of the present invention are used in methods to treat a variety of diseases. Any disease in which cells express a specific protease or other cleaving agents, or diseases in which a specific protease may be delivered to the target cell, are amenable to treatment with the compositions. Generally, the methods of treatment comprise administering a therapeutically effective amount of a composition, where the cargo or compound of interest is a therapeutic compound, and where the composition is capable of being converted to a cell penetrating form, thereby facilitating delivery of the therapeutic compound into the target cell.

Inflammatory Disorders. In one aspect, the compositions are used to treat inflammatory disorders. In one embodiment, the compositions are used to treat inflammation resulting from cerebral ischemia. Degradation of basal lamina during ischemia is most extensive in the region where injury is maximal. Disruption in the microvascular basal lamina occurs when secreted proteases, such as metalloproteinases and plaminogen activators degrade laminin, collagen and fibronectin. Serine protease activated by proteolysis further the remodeling process. In addition, polymorphonuclear cell granule enzymes, including collagenase, gelatinase, elastase and cathepsin are released during the inflammatory phase following ischemia.

For purposes of controlled delivery of a compound of interest into the cells involved in the inflammatory process, the cleavage site is comprised of sequences recognized by proteases activated during the inflammatory reaction. As described above, the cleavages sites may comprise those recognized and acted upon, by way of example and not limitation, collagenase, gelatinase, elastase, cathepsins, MMP-2, MMP-9 and the like.

For example, the cleavage site for compositions used for treating inflammatory conditions may comprise a cleavage sequence found on protease activated receptors (PARS). PARS are part of the family of G coupled receptors and are involved in the inflammatory response. Four types of PAR, termed PAR₁-PAR₄ have been identified. The receptors are proteolytically activated by inflammatory related proteases, such as thrombin, granzyme A, cathepsin G, trypsin, and coagulation factor Xa. Cleavage unmasks a tethered region on the receptor that interacts with the receptor, thereby initiating signal transduction events leading to inflammation. By using a cleavage site recognized by proteases involved in proteolysis of PAR, the delivery of therapeutic agents into cells can be directed to cells residing near the site of the inflammatory process, and limit the extent of the inflammatory reaction.

The therapeutic compound can comprise a compound that inhibits synthesis of cellular products mediated by activation of the PARS. For example, expression of ICAM-1 in endothelial cells is stimulated by thrombin mediated proteolysis of PAR1. Transcription of ICAM-1 is regualated by NKkB. Thus, a peptide inhibitor of NFkB may be delivered selectively to endothelial cells to inhibit ICAM-1 synthesis. Since interaction of ICAM-1 with its counter receptors on the surface of leukocytes is vital to PMN adhesion and tranendothelial migration, inhibition of ICAM-1 synthesis can reduce adhesion of polymorphonuclear lymphocytes, thereby reducing furtherance of the inflammatory response.

Treatments for Tumors and Metastasis. Degradation of the extracellular matrix is a hallmark of tumor invasion and metastasis. Most of this degradation is mediated by matrix metalloproteinases (MMPs), a family of enzymes that, collectively, degrades the extracellular matrix. For example, two matrix metalloproteinases (MMPs) Mr 72,000 type IV collagenase (MMP-2, gelatinase A) and Mr 92,000 type IV collagenase (MMP-9, gelatinase B) play key roles in tissue remodeling and tumor invasion by digestion of extracellular matrix barriers. Expression of matrix metalloproteinase 3 (MMP-3, stromelysin-1) is detected in 89.7% of breast tumours and correlates with presence of p53 tumour suppressor gene product (Ioachim, E. E., Anticancer Res. 18(3A): 1665-1670 (1998))

Interstitial collagenases, a subfamily of MMPs that cleaves the stromal collagens types I and III, comprised of collagenase 1 (MMP-1), collagenase 3 (MMP-13), and the MT-MMPs, membrane-bound MMPs are also expressed in a wide variety of advancing tumors. Collagenases can mediate tumor invasion through several mechanisms, which include constitutive production of enzyme by the tumor cells, induction of collagenase production in the neighboring stromal cells, and interactions between tumor/stromal cells to induce collagenase production by one or both cell types. Expression of the interstitial collagenases is associated with a poor prognosis in a variety of cancers (Brinckerhoff, C. E., Clin Cancer Res. 6(12):4823-4830 (2000)). MMP-1 3 is primarily expressed by myofibroblasts in human breast carcinoma and expression in Ductal Carcinoma I S lesions often is associated with microinvasive events, suggesting an essential role for MMP-13 during transition of DCIS lesions to invasive ductal carcinomas (Nielsen, B. S., Cancer Res. 61(19):7091-100 (2001)).

Expression of the matrix metalloprotease pump-1 gene (also referred to as MMP-7, Matrilysin) is significantly elevated in ovarian tumors having either low malignant potential tumors and carcinomas. The pump-1 transcript was abundant in carcinoma but seldom expressed in normal adult tissues including normal ovary. Expression of the protease may contribute to its invasive nature or growth capacity (Tanimoto, H., Tumour Biol. 20(2):88-98 (1999)).

Similarly, cathepsin D is detected in a substantial majority of colorectal cases and is correlated with p53 protein and pRb expression (Ioachim, E. E., Anticancer Res. 19(3A):2147-55 (1999)).

Human FAP is also selectively expressed by tumor stromal fibroblasts in epithelial carcinomas, but not by epithelial carcinoma cells, normal fibroblasts, or other normal tissues. FAP has been shown to have both in vitro dipeptidyl peptidase and collagenase activity, HT-29 xenografts treated with these inhibitory anti-FAP antisera exhibited attenuated growth compared with tumors treated with preimmunization rabbit antisera. These data demonstrate the ability of FAP to potentiate tumor growth in an animal model. Moreover, tumor growth is attenuated by antibodies that inhibit the proteolytic activity of FAP. These findings suggest a possible therapeutic role for functional inhibition of FAP activity (Cheng, J. D., Cancer Res. 62(16):4767-72 (2002)).

Extraxcellular proteinases in skin cancer. Different forms of skin cancer are characterized by the expression of specific patterns of extracellular proteinases. Activity of serine proteinases such as u-PA and t-PA has been used for classification and prognosis of skin cancer (Maguire et al., Int J Cancer 85(4):457-9 (2000); Ferrier et al., Br J Cancer 83(10):1351-9 (2000)).

Increased activity of another group of proteinases (matrix metalloproteinases; MMPs) has also been described in skin cancer. Degradation of basement membranes and extracellular matrix is an essential step in skin cancer cell migration, invasion and metastasis formation. Matrix metalloproteinases and their inhibitors play a crucial role in these complex multistep processes. Skin cancer cells express a number of matrix metalloproteinase family members such as MMP-1, MMP-2, MMP-9, MMP-13, MT I-MMP and others (Dumas et al., Anticancer Res. 19(4B):2929-38 (1999); Walker R. A. and Woolley, D. E. Virchows Arch. 435(6):574-9 (1999); Airola, K, and Fusenig, N.E., J Invest Dermatol. 116(1):85-92 (2001); Bodey, et al., In Vivo 15(1):57-64 (2001); Kerkela et al., Br J Cancer 84(5):659-69 (2001); Papathoma et al., Mol. Carcinog. 31(2):74-82 (2001)) as well as their tissue inhibitors TIMP-1, TIMP-2 and TIMP-3 (for review see Hofmann et al., J. Invest. Dermatol. 115(3):33744 (2000)).

An exemplary treatment of skin cancer in the present invention utilizes peptides that affect function of several tumor type specific transcription factors. Peptides are delivered topically or using a patch in the form of inactive molecules that will be converted into active molecules by extracellular proteinases that are present at high levels in cancerous tissue but not in normal skin. Activated drug contains cell penetrating peptide that is responsible for the internalization of the drug. Therapeutic part of the peptide mimics functional domain of skin cancer type specific transcription factors. The patch contains several of these peptides which all target different transcription factors, and their combined action blocks proliferation, induces differentiation, or induces apoptosis of skin cancer cells

Periodontitis. Evidence of the role of matrix metalloproteinases (MMPs) produced by resident and inflammatory cells in periodontal destruction is now well established. The imbalance between MMPs and TIMPs is associated with the pathologic breakdown of the extracellular matrix during periodontitis (Seguier, S. et al., J Periodontol. 72(10):1398-406 (2001)). For example, collagenase expression is elevated in gingival fibroblasts from periodontitis patients, and is correlated with expression of c-fos and egr-1, two transcription factor known to regulate expression of metalloproteases (Trabandt, A. et al., J. Oral Pathol. Med. 21(5):232-240 (1992)). Moreover, NF-kB-like DNA binding activity is induced in gingival fibroblasts by IL-1 and is known to regulate genes involved in inflammatory process. Thus, a composition for modulating periodontitis associated tissue destruction would have a cleavage site for collagenase and further contain a modulator of NFkB, c-fos, or egr-1 transcription factors.

Asthma. Activation of mast cells by crosslinking of IgE receptors results in release of granule associated mediators, which include proteases chymase and typtase. The enzymes are believed to exert tissue remodeling in allergic asthma and regulate cell signaling events.

The increased in MMP-9 production and activity observed in the present study suggests a process of extracellular matrix degradation in acute severe asthmatic patients and proposes MMP-9 as a non-invasive systemic marker of inflammation and airway remodelling in asthma (Belleguic, C. Clin Exp Allergy 32(2):217-23 (2002)).

Pharmaceutical Compositions and Administration

Pharmaceutical Compositions

The compounds of the present invention can be formulated as pharmaceutical compositions. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form will vary depending upon the patient and the particular mode of administration.

Administration and Dose

The concentrations of the peptides or nucleic acid encoding therefore will be determined empirically in accordance with conventional procedures for the particular purpose. Generally, for administering the peptides ex vivo or in vivo for therapeutic purposes, the subject peptides are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms or manifestations of the disorder or disease.

The compositions of the present invention can be administered by a variety of methods, including, for example, orally, enterally, mucosally, percutaneously, or parenterally. Parenteral administration may be by intravenous, intramuscular, subcutaneous, intracutaneous, intraarticular, intrathecal, and intraperitoneal infusion or injection, including continuous infusions or intermittent infusions with pumps available to those skilled in the art. Administration of the pharmaceutical compositions may be through a single route or concurrently by several routes. For instance, oral administration can be accompanied by intravenous or parenteral injections.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider includes whether the disease is acute or chronic, relapsing or remitting phase, and the progressiveness of the disease. Determining the dosages and times of administration for a therapeutically effective amount are well within the skill of the ordinary person in the art.

For any compounds used in the present invention, therapeutically effective dose is readily determined by methods well known in the art. For example, an initial effective dose can be estimated initially from cell culture assays. A dose can then be formulated in animal models to generate a circulating concentration or tissue concentration, including that of the IC50 as determined by the cell culture assays.

In addition, the toxicity and therapeutic efficacy are generally determined by cell culture assays and/or experimental animals, typically by determining a LD50 (lethal dose to 50% of the test population) and ED50 (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein.

Generally, in the case where a peptide composition is administered directly to a host, the present invention provides for a bolus or infusion of the subject composition that will administered in the range of about 0.01-50, more usually from about 0.1-25 mg/kg body weight of host. The amount will generally be adjusted depending upon the half-life of the peptide. Formulations for administration may be presented in unit a dosage form, e.g., in ampules, capsules, pills, or in multidose containers or injectables. Dosages in the lower portion of the range and even lower dosages may be employed, where the peptide has an enhanced half-life or is provided as a depot, such as a slow release composition comprising particles, a polymer matrix which maintains the peptide over an extended period of time (e.g., a collagen matrix, carbomer, etc.), use of a pump which continuously infuses the peptide over an extended period of time with a substantially continuous rate, or the like. The dose is also adjusted in relation to the route of administration. Thus for example, if the administration is systemic, either oral or intravenous, the dose is appropriately adjusted for bioavailability, as compared to more targeted delivery, such as by topical or transdermal route. The host or subject may be any mammal including domestic animals, pets, laboratory animals, primates, particularly human subjects.

In addition to administering the subject peptide compositions directly to a cell culture in vitro, to particular cells ex vivo, or to a mammalian host in vivo, nucleic acid molecules (DNA or RNA) encoding the subject compositions may also be administered thereto, thereby providing an effective source of the subject peptides for the application desired. As described above, nucleic acid molecules encoding the subject peptides may be cloned into any of a number of well known expression plasmids (Sambrook et al., supra) and/or viral vectors, preferably adenoviral or retroviral vectors (see for example, Jacobs et al., J. Virol. 66:2086-2095 (1992), Lowenstein, Bio/Technology 12:1075-1079 (1994) and Berkner, Biotechniques 6:616-624 (1988)), under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hydrogels), and the like, or, in the case of viral-based vectors, by systemic administration. Tissue specific promoters may be optionally employed, assuring that the peptide of interest is expressed only in a particular tissue or cell type of choice. Methods for recombinantly preparing such nucleic acid-based vehicles are well known in the art, as are techniques for administering nucleic acid-based vehicles for peptide production.

Transdermal Delivery

The preparation of suitable transdermal delivery systems is described e.g. in WO 92/21334, WO 92/21338 and EP 413487. Such system may comprise (1) a drug impermeable backing layer and (2) an adhesive layer that fixes the bandage to the skin, wherein the composition is dispersed in the adhesive layer. Alternatively, the system may comprise (1) a drug impermeable backing layer, (2) an adhesive layer and (3) a matrix layer preferably made of a polymer material in which the drug is dispersed. The release rate of the therapeutic compound from the device is typically controlled by the polymer matrix. The system may also comprise (1) a drug impermeable backing layer, (2) an adhesive layer, (3) a drug permeable membrane sealed to one side of said backing layer as to define at least one drug reservoir compartment therebetween, and (4) a drug or composition thereof within said drug reservoir. In this case the drug in the reservoir is usually in liquid or gel form. The drug permeable membrane controls the rate at which the drug is delivered to the skin.

Iontophoretic transdermal delivery systems according to known technology can also be used in the transdermal delivery of levosimendan. Term “iontophoresis” means using small electric current to increase trans-dermal permeation of charged drugs. The method is reviewed in e.g., Burnette R., Iontophoresis. In Transdermal Drug Delivery, pp. 247-292, Eds. Guy, R. and Hadgraft, J., Marcel Dekker Inc., New York and Baselm (1989). Iontophoretic transdermal delivery system typically include a first (donor) electrode containing an electrolytically available active compound within a suitable vehicle or carrier, a second (passive) electrode and a power source, the first and second electrodes each being in electrically conductive communication with the power source. The first and second electrodes are being adapted for spaced apart physical contact with the skin whereby, in response to a current provided by the power source through the electrodes, a therapeutic amount of the active compound is administered through the skin to a patient.

Suitable skin penetration enhancers include those well known in the art, for example, C₂-C₄ alcohols such as ethanol and isopropanol; surfactants, e.g. anionic surfactants such as salts of fatty acids of 5 to 30 carbon atoms, e.g., sodium lauryl sulphate and other sulphate salts of fatty acids, cationic surfactants such as alkylamines of 8 to 22 carbon atoms, e.g., oleylamine, and nonionic surfactants such as polysorbates and poloxamers; aliphatic monohydric alcohols of 8 to 22 carbon atoms such as decanol, lauryl alcohol, myristyl alcohol, palmityl alcohol, linolenyl alcohol and oleyl alcohol; fatty acids of 5 to 30 carbon atoms such as oleic acid, stearic acid, linoleic acid, palmitic acid, myristic acid, lauric acid and capric acid and their esters such as ethyl caprylate, isopropyl myristate, methyl laurate, hexamethylene palmitate, glyceryl monolaurate, polypropylene glycol monolaurate and polyethylene glycol monolaurate; salicylic acid and its derivatives; alkyl methyl sulfoxides such as decyl methyl sulfoxide and dimethyl sulfoxide; 1-substituted azacycloalkan-2-ones such as 1-dodecylazacyclo-heptan-2-one sold under the trademark AZONE; amides such as octylamide, oleicamide, hexamethylene lauramide, lauric diethanolamide, polyethylene glycol 3-lauramide, N,N-diethyl-m-toluamide and crotamiton; and any other compounds compatible with levosimendan and the packages and having transdermal permeation enhancing activity.

### EXPERIMENTAL

### Example 1

### Effect of CPP-Mimicking Peptides on Proliferation and Apoptosis of melanoma Cells

The ability to block interaction of MITF, SOX10 and STAT3 with active transcriptional complex and inhibit proliferation and stimulate apoptosis of melanoma cells were tested on human melanoma cell lines SK-MEL-28 and WM 266-4, and mouse melanoma cell line B16.

Peptides. Cell penetrating peptides were generated by combining peptides that mimic interaction domains of MITF, SOX10 and STAT3 (bold) to nuclear localization signal and cell penetrating sequence (italics).
MITF-int1: RPKKRKVRRRFNINDRIKELGTLIPKSNDPDMRWN
SOX10-int1: RPKKRKVRRRVKRPMNAFMVWAQAARRKLADQY
STAT3-int1: RPKKRKVRRRKMQQLEQMLTALDQMRRSIVSELAGLLS
Scr-int1: RPKKRKVRRRQLMLEPYALDMSRIRVLSESLGLATQSG (control)

Methods. Human melanoma cell lines SK-MEL-28 and WM 2664 and mouse melanoma cell line B16 were obtained from the American Tissue Culture Collection (ATCC). Cells were cultured according to recommendations of ATCC (DMEM, 10% FCS, penicillin+streptomycin) and used in experiments after two passages in the laboratory. Cells were grown in 24 well plates, each treatment in triplicates. Cells were plated 16 hours prior treatments started. Peptides were added to the media, and media was changed every day during 7 day experiment. CPP concentration was 10 μM

For cell counting, cells were trypsinized (0.25% Trypsin, 2 mM EDTA) in Ca⁺², Mg⁺² free PBS. Cells were precipitated and resuspended in 100 μl of PBS, and 5 μl were removed for counting

WST-1 test was performed to measure mitochondrial activity, which can also be looked as a measure of cell number.

Apoptosis was analyzed using Biovision Annexin V-Cy3 Apoptosis Kit according to manufacturers protocols.

TABLE 1
Effect of CPP mimicking peptides on proliferation and apoptosis

| | | | Cell Count | | WST-1 | Apoptosis |
|---|---|---|---|---|---|---|
| Cell line | Peptide | conc | start | 7 d | 7 d | 7 d/% |
| SK-MEL-28 | no | | 10 | 52 | 1 | 0 |
| | MITF-int1 | 10 mM | 10 | 39 | 0.7 | 35 |
| | SOX10-int1 | 10 mM | 10 | 28 | 0.51 | 55 |
| | STAT3-int1 | 10 mM | 10 | 40 | 0.79 | 29 |
| | Scr-int1 | 10 mM | 10 | 53 | 1 | 0 |
| | MITF + SOX10 | 10 mM | 10 | 18 | 0.39 | 62 |
| | MITF + STAT3 | 10 mM | 10 | 20 | 0.28 | 78 |
| | SOX10 + STAT3 | 10 mM | 10 | 21 | 0.34 | 67 |
| | MITF + SOX10 + STAT3 | 10 mM | 10 | 5 | 0.1 | 95 |
| WM 266-4 | no | 10 mM | 10 | 39 | 1 | 0 |
| | MITF-int1 | 10 mM | 10 | 30 | 0.8 | 21 |
| | SOX10-int1 | 10 mM | 10 | 25 | 0.6 | 31 |
| | STAT3-int1 | 10 mM | 10 | 34 | 93 | 10 |
| | Scr-int1 | 10 mM | 10 | 41 | 1 | 0 |
| | MITF + SOX10 | 10 mM | 10 | 19 | 0.5 | 47 |
| | MITF + STAT3 | 10 mM | 10 | 13 | 0.6 | 58 |
| | SOX10 + STAT3 | 10 mM | 10 | 8 | 0.21 | 81 |
| | MITF + SOX10 + STAT3 | 10 mM | 10 | 3 | 0.1 | 93 |
| B16 | no | 10 mM | 10 | 80 | 1 | 0 |
| | MITF-int1 | 10 mM | 10 | 63 | 85 | 16 |
| | SOX10-int1 | 10 mM | 10 | 72 | 88 | 12 |
| | STAT3-int1 | 10 mM | 10 | 59 | 72 | 28 |
| | Scr-int1 | 10 mM | 10 | 83 | 1.1 | 0 |
| | MITF + SOX10 | 10 mM | 10 | 45 | 52 | 53 |
| | MITF + STAT3 | 10 mM | 10 | 35 | 42 | 61 |
| | SOX10 + STAT3 | 10 mM | 10 | 31 | 32 | 73 |
| | MITF + SOX10 + STAT3 | 10 mM | 10 | 2 | 2 | 98 |

Results. Peptides derived from MITF, SOX10 and STAT3 transcription factors inhibit proliferation and induce apoptosis in melanoma cell lines in vitro. The effect of mimicking peptides is additive such that treatment with three peptides inhibits proliferation more that 90% and induces apoptosis in approxiamtely 95% of cells. Use of a mixture of mimicking peptides that affect several transcription factor (TF( systems is more efficient than using just one inhibitor molecule that blocks effect of transcription factors completely. Using suboptimal level of several drugs that target specific but different pathways results in specific and effective treatment, whereas side effects are minimal since effectiveness depends on the activity of pathways in specific cell type.

### Example 2

### Analysis of Mimicking Peptides with Inhibited Cell Penetrating (CPP) Activity

Peptides MITF-Int1, SOX10-Int1 and STAT3-Int1 were modified so that the cell penetrating activity was blocked by the inhibitory peptide sequence that included a stretch of amino acids that formed a recognition site for MMP2 and MMP9 (underlined).

These peptides will be converted into active cell penetrating peptides followed by the cleavage of inhibitory sequences by extracellular proteinases MMP2 and MMP9. These matrix metalloproteases are present at high levels in the extracellular matrix of melanoma cells but not normal skin cells such that these modified peptides will be taken into the melanoma but not normal skin (keratinocytes) cells.

Peptide compositions. Peptides were as follows:
MITF-int1M: TTGGSSPQGLEAKRPKKRKVRRRFNINDRIKELGTLIPKSNDPDMRWN
SOX10-int1M: TTGGSSPQGLEAKRPKKRKVRRRVKRPMNAFMVWAQAARRKLADQY
STA3-int1: TTGGSSPQGLEAKRPKKRKVRRRKMQQLEQMLTALDQMRRSIVSELAGLLS
Scr-int1M: TTGGSSPQGLEAKRPKKRKVRRRQLMLEPYALDMSRIRVLSESLGLATQSG (control)
   where the underlined residues correspond to the inhibitor of cell penetrating peptide, the italicized residues correspond to the cell penetrating peptide, and the bolded residues correspond to transcription factor inhibitor peptide.

Methods. Human melanoma cell lines SK-MEL-28 and WM 266-4 were obtained from the American Tissue Culture Collection (ATCC) and were cultured according to recommendations of ATCC (DMEM, 10% FCS, penicillin+streptomycin). Human keratinocytes were obtained from Clonetics and cultured according to manufacturers protocol. Cells were used in experiments after two passages in the laboratory. Cells were grown in 24 well plates, each treatment in triplicates. Cells were plated 16 hours prior treatments started. Peptides were added to the media, and media was changed every day during 7 day experiment. CPP concentration was 10 μM.

For cell counting, cells were trypsinized (0.25% Trypsin, 2 mM EDTA) in Ca, Mg free PBS. Cells were precipitated and resuspended in 100 μl of PBS, and 5 μl were removed for counting.

WST-1 test was performed to measure mitochondrial activity, which can also be used as a measure of cell number.

Apoptosis was analyzed using Biovision Annexin V-Cy3 Apoptosis Kit according to manufacturers protocols.

TABLE 2
Effect of modified CPP mimicking peptides on proliferation and apoptosis

| | | | Cell Count | | WST-1 | Apoptosis |
|---|---|---|---|---|---|---|
| Cell line | Peptide | conc | Start | 7 d | 7 d | 7 d/% |
| SK-MEL-28 | no | | 10 | 50 | 1 | 0 |
| | MITF-int1M | 10 mM | 10 | 42 | 0.81 | 20 |
| | SOX10-int1M | 10 mM | 10 | 35 | 72 | 32 |
| | STAT3-int1M | 10 mM | 10 | 42 | 0.84 | 12 |
| | Scr-int1M | 10 mM | 10 | 48 | 1 | 0 |
| | MITFM + SOX10M | 10 mM | 10 | 23 | 0.49 | 51 |
| | MITFM + STAT3M | 10 mM | 10 | 28 | 56 | 42 |
| | SOX10M + STAT3M | 10 mM | 10 | 22 | 0.37 | 61 |
| | MITFM + SOX10M + STAT3M | 10 mM | 10 | 9 | 0.19 | 91 |
| WM 266-4 | no | | 10 | 32 | 1 | 0 |
| | MITF-int1M | 10 mM | 10 | 25 | 0.8 | 21 |
| | SOX10-int1M | 10 mM | 10 | 22 | 0.7 | 32 |
| | STAT3-int1M | 10 mM | 10 | 29 | 91 | 13 |
| | Scr-int1M | 10 mM | 10 | 30 | 1 | 0 |
| | MITFM + SOX10M | 10 mM | 10 | 18 | 0.6 | 44 |
| | MITFM + STAT3M | 10 mM | 10 | 12 | 0.45 | 63 |
| | SOX10M + STAT3M | 10 mM | 10 | 8 | 0.2 | 80 |
| | MITFM + SOX10M + STAT3M | 10 mM | 10 | 5 | 0.17 | 90 |
| keratinocyte | no | | 10 | 40 | 1 | 0 |
| | MITF-int1M | 10 mM | 10 | 38 | 93 | 5 |
| | SOX10-int1M | 10 mM | 10 | 37 | 93 | 6 |
| | STAT3-int1M | 10 mM | 10 | 41 | 1 | 2 |
| | Scr-int1M | 10 mM | 10 | 40 | 1 | 0 |
| | MITFM + SOX10M | 10 mM | 10 | 36 | 91 | 10 |
| | MITFM + STAT3M | 10 mM | 10 | 35 | 89 | 12 |
| | SOX10M + STAT3M | 10 mM | 10 | 33 | 84 | 19 |
| | MITFM + SOX10M + STAT3M | 10 mM | 10 | 34 | 85 | 17 |

Modified mimicking peptides suppress proliferation and induce apoptosis only in melanoma cells whereas normal keratinocytes show very little response.

### Example 3

### Analysis of the Effect of Mimicking Peptides on the Activity of Dopacrome Tautomerase (Dct/Trp2) Using Transient CAT Assay

SOX10 and MITF interact with the proximal promoter of Dct/Trp2 gene and induces its activity (Ludwig A. et al., FEBS Lett. 556 (1-3):236-244 (2004)). Thus, a Dct/Trp2 proximal promoter reporter construct was used to analyze effect of mimicking peptides on promoter activity using transient CAT assay.

Methods. Human melanoma cell lines SK-MEL-28 and WM 266-4 were obtained from the American Tissue Culture Collection (ATCC) and were cultured according to recommendations of ATCC (DMEM, 10% FCS, penicillin+streptomycin). Dct/Trp2 proximal promoter-CAT construct (Ludwig et al., supra) was used in all experiments.

Cells were transfected by using FuGene reagent (Roche Molecular Biochemicals) according to manufacturer's instructions. Freeze-thaw lysates of cells collected 48 h after the transfection were assayed for CAT activity as described (Pothier, F. et al., DNA Cell Biol. 11(1):83-90 (1992)). At least two different DNA preparations were tested for each plasmid. To normalize the transfection efficiencies, cells were cotransfected with pON260 expressing β-galactosidase (Spaete, R. R. and Mocarski, E. S., J. Virol. 54(3):817-24 (1985); Spaete, R. R. and Mocarski, E. S., J. Virol. 56(1):13543 (1985)). All the CAT activities were normalized to total protein and β-galatosidase activity.

Peptide compositions: Peptides were as follows.
MITF-int1: RPKKRKVRRRFNINDRIKELGTLIPKSNDPDMRWN
SOX10-int1: RPKKRKVRRRVKRPMNAFMVWAQAARRKLADQY
STAT3-int1: RPKKRKVRRRKMQQLEQMLTALDQMRRSIVSELAGLLS
Scr-int1: RPKKRKVRRRQLMLEPYALDMSRIRVLSESLGLATQSG (control)
   where the italicized residues correspond to the cell penetrating peptide and the bolded residues correspond to the inhibitor of the transcription factor inhibitor peptide.

Following transfection, cells were grown in 6 well plates, each treatment in triplicates. Peptides were added to the media and media was changed every day during the 7 day experiment. CPP concentration was 10 μM.

Results. CAT assay data clearly show that in both melanoma cell lines, MITF and SOX10 mimicking peptides suppress Dct/Trp2 promoter activity significantly whereas control and STAT3 peptides do not have significant effect.

### Example 4

### Analysis of the Effect of Modified Mimicking Peptides on the Growth of Melanomas Using Mouse Tumor Xenograft Model

The effect of modified mimicking peptides was analyzed by inducing melanomas in mouse skin by grafting suspension of melanoma cells. The membrane patch which contained peptides was placed directly on top of the skin exhibiting melanoma and affixed with adhesive bandages. Tumor diameter was measured 1, 2, 3, 5, 7 days after treatmet started.

Methods. Mouse melanoma cell line B16 was cultured as described above, and approximately 5×10⁶ cells were injected subcutaneously into the left and right limbs of three C57BL/6JOIaHsd mice. After 4 days, when melanomas were approximately 2 mm in diameter, the membrane patches, as described in Example 1, were placed directly on top of the skin exhibiting melanoma and affixed with adhesive bandages.

Peptide compositions. Peptids were as follows.
MITF-int1M: TTGGSSPQGLEAKRPKKRKVRRRFNINDRIKELGTLIPKSNDPDMRWN
SOX10-int1M: TTGGSSPQGLEAKRPKKRKVRRRVKRPMNAFMVWAQAARRKLADQY
STAT3-int1: TTGGSSPQGLEAKRPKKRKVRRRKMQQLEQMLTALDQMRRSIVSELAGLLS
Scr-int1M: TTGGSSPQGLEAKRPKKRKVRRRQLMLEPYALDMSRIRVLSESLGLATQSG (control)
   where the underlined residues correspond to the inhibitor of cell penetrating peptide, the italicized residues correspond to the cell penetrating peptide, and the bolded residues correspond to transcription factor inhibitor peptide.

Administration. Cellulose membrane was immersed in a solution of peptides (100 μM). Size of tumor was measured 1, 2, 3, 5 and 7 days following patch treatment. At the same time new patch was applied. All experiments were done in triplicates (3 animals per group).

TABLE 3
Effect of modified mimicking peptides on tumor growth

| | tumor size change (%) | | | | | |
|---|---|---|---|---|---|---|
| peptide | start | 1 d | 2 d | 3 d | 5 d | 7 d |
| no treatment | 100 | 110 | 150 | 170 | 200 | 260 |
| MITF-int1M | 100 | 112 | 135 | 150 | 170 | 210 |
| SOX10-int1M | 100 | 111 | 127 | 146 | 171 | 224 |
| STAT3-int1M | 100 | 109 | 124 | 154 | 181 | 217 |
| Scr-int1M | 100 | 109 | 147 | 175 | 211 | 272 |
| MITFM + SOX10M | 100 | 111 | 121 | 133 | 129 | 134 |
| MITFM + STAT3M | 100 | 110 | 115 | 119 | 123 | 130 |
| SOX10M + STAT3M | 100 | 109 | 120 | 123 | 141 | 158 |
| MITFM + SOX10M + | 100 | 110 | 112 | 119 | 121 | 120 |
| STAT3M | | | | | | |

Results. Results of animal experiments show that modified peptides inhibit tumor growth when delivered using a transdermal patch. Individual peptides had a significant effect on tumor growth, but the combination of 3 peptides almost completely blocked the growth of tumor.

The descriptions of specific embodiments herein are presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

We claim:

1. A composition for the controlled delivery of a compound of interest into a target cell, comprising:
(i) a cell penetrating peptide;
(ii) a cell penetrating peptide inhibitor;
(iii) a compound of interest; and
(iv) a cleavage site;
wherein said cell penetrating peptide inhibitor inhibits the translocation activity of said cell penetrating peptide, wherein cleavage at said cleavage site by a cleaving agent disinhibits said cell penetrating peptide, and wherein the disinhibited cell penetrating peptide is capable of translocating said compound of interest into said target cell.

2. The composition according to claim 1, further comprising a subcellular targeting sequence.

3. The composition according to claim 1, wherein said compound of interest is a reporter molecule.

4. The composition according to claim 1, wherien said compound of interest is a therapeutic agent.

5. The composition according to claim 1, wherein said cleavage site is a recognition site for a matrix metalloprotease.

6. The composition according to claim 1, wherein said cell penetrating peptide inhibitor comprises said cleavage site.

7. The composition according to claim 2, wherein said cell penetrating peptide comprises said subcellular targeting sequence.

8. The composition according to claim 1, wherein said compound of interest is a nucleic acid.

9. The composition according to claim 8, wherein said nucleic acid is an siRNA.

10. A method for the controlled delivery of a compound of interest into a target cell, comprising contacting said target cell with a composition, said composition comprising:
(i) a cell penetrating peptide;
(ii) a cell penetrating peptide inhibitor;
(iii) a compound of interest; and
(iv) a cleavage site;
wherein said cell penetrating peptide inhibitor inhibits the translocation activity of said cell penetrating peptide, wherein cleavage at said cleavage site by a cleaving agent disinhibits said cell penetrating peptide, wherein the disinhibited cell penetrating peptide is capable of translocating said compound of interest into said target cell, and wherein said cleaving agent is present in the vicinity of said target cell.

11. The method according to claim 10, wherein said composition further comprises a subcellular targeting sequence.

12. The method according to claim 10, wherein said compound of interest is a reporter molecule.

13. The method according to claim 10, wherein said compound of interest is a therapeutic agent.

14. The method according to claim 10, wherein said cleavage site is a recognition site for a matrix metalloprotease.

15. The method according to claim 10, wherein said cell penetrating peptide inhibitor comprises said cleavage site.

16. The method according to claim 10, wherein said cell penetrating peptide comprises said subcellular targeting sequence.

17. The method according to claim 10, wherein said compound of interest is a nucleic acid.

18. The method according to claim 17, wherein said nucleic acid is an siRNA.


SEQUENCE LISTING (62 sequences)

SEQ ID NO: 1 - protein, 10 aa, Homo sapiens
RPKKRKVRRR

SEQ ID NO: 2 - protein, 13 aa, Homo sapiens
TTGGSSPQPLEAP

SEQ ID NO: 3 - protein, 13 aa, Homo sapiens
TTGGSSPQGLEAK

SEQ ID NO: 4 - protein, 16 aa, Apis mellifera
RQIKIWFQNRRMKWKK

SEQ ID NO: 5 - protein, 26 aa, Kaposi′s sarcoma-associated herpesvirus
AAVALLPAVLLALLAPAAADQNQLMP

SEQ ID NO: 6 - protein, 11 aa, Human immunodeficiency virus type 1
YGRKKRRQRRR

SEQ ID NO: 7 - protein, 9 aa, Human immunodeficiency virus type 1
RKKRRQRRR

SEQ ID NO: 8 - protein, 34 aa, human herpesvirus 1
DAATATRGRSAASRPTERPRAPARSASRPRRPVE

SEQ ID NO: 9 - protein, 16 aa, Homo sapiens
RRWRRWWRRWWRRWRR

SEQ ID NO: 10 - protein, 7 aa, Homo sapiens
RRRRRRR

SEQ ID NO: 11 - protein, 11 aa, Homo sapiens
KKKKKKKKGGC

SEQ ID NO: 12 - protein, 11 aa, Homo sapiens
KWKKKWKKGCC

SEQ ID NO: 13 - protein, 11 aa, Homo sapiens
RWRRRWRRGGC

SEQ ID NO: 14 - protein, 28 aa, Artificial
  Synthetic
GALFLGFLGGAAGSTMGAWSQPKSKRKV

SEQ ID NO: 15 - protein, 21 aa, Artificial
  Synthetic
AGYLLGKINLKALAALAKKIL

SEQ ID NO: 16 - protein, 26 aa, Homo sapiens
DPKGDPKGVTVTVTVTVTGKGDPKPD

SEQ ID NO: 17 - protein, 18 aa, Homo sapiens
KLALKLALKALKAALKLA

SEQ ID NO: 18 - protein, 18 aa, Mus musculus
LLIILRRRIRKQAHAHSK

SEQ ID NO: 19 - protein, 16 aa, Rattus norvegicus
RVIRVWFQNKRCKDKK

SEQ ID NO: 20 - protein, 21 aa, Homo sapiens
KETWWETWWTEWSQPKKKRKV

SEQ ID NO: 21 - protein, 28 aa, Mus musculus
MANLGYWLLALFVTMWTDVGLCKKRPKP

SEQ ID NO: 22 - protein, 7 aa, Homo sapiens
PLGLWAR

SEQ ID NO: 23 - protein, 4 aa, Homo sapiens
RPGL

SEQ ID NO: 24 - protein, 7 aa, Homo sapiens
PQGIAGQ

SEQ ID NO: 25 - protein, 7 aa, Artificial
  Synthetic
XXXXXXX

SEQ ID NO: 26 - protein, 8 aa, Homo sapiens
RPLALWRS

SEQ ID NO: 27 - protein, 7 aa, Homo sapiens
PLAYWAR

SEQ ID NO: 28 - protein, 6 aa, Homo sapiens
PGLWAR

SEQ ID NO: 29 - protein, 7 aa, Homo sapiens
PLACWAR

SEQ ID NO: 30 - protein, 4 aa, Artificial
  Synthetic
SSXY

SEQ ID NO: 31 - protein, 6 aa, Artificial
  Synthetic
IEXDXG

SEQ ID NO: 32 - protein, 6 aa, Artificial
  Synthetic
XXXSRA

SEQ ID NO: 33 - protein, 5 aa, Artificial
  Synthetic
XXSRA

SEQ ID NO: 34 - protein, 4 aa, Artificial
  Synthetic
RXXR

SEQ ID NO: 35 - protein, 7 aa, Artificial
  Synthetic
XXXXXXX

SEQ ID NO: 36 - protein, 10 aa, Homo sapiens
LPDRSFLLRN

SEQ ID NO: 37 - protein, 8 aa, Homo sapiens
LPIKTFRG

SEQ ID NO: 38 - protein, 10 aa, Homo sapiens
  MISC_FEATURE (5)..(5): The Xaa at position 5 is isoleucine or valine
DAVYXHPFHL

SEQ ID NO: 39 - protein, 9 aa, Homo sapiens
SKGRSLIGR

SEQ ID NO: 40 - protein, 5 aa, Homo sapiens
KFERQ

SEQ ID NO: 41 - protein, 36 aa, Homo sapiens
MLIPIAGFFALAGLVLIVLIAYLIGRKRSHAGYQTI

SEQ ID NO: 42 - protein, 35 aa, Homo sapiens
LVPIAVGAALAGVLILVLLAYFIGLKHHHAGYEQF

SEQ ID NO: 43 - protein, 27 aa, Homo sapiens
MLRTSSLFTRRVQPSLFSRNILRLQST

SEQ ID NO: 44 - protein, 25 aa, Homo sapiens
MLSLRQSIRFFKPATRTLCSSRYLL

SEQ ID NO: 45 - protein, 59 aa, Homo sapiens
MFSMLSKRWAQRTLSKSFYSTATGAASKSGKLTQKLVTAGVAAAGITASTLLYADSLTA

SEQ ID NO: 46 - protein, 41 aa, Homo sapiens
MKSFITRNKTAILATVAATGTAIGAYYYYNQLQQQQQRGKK

SEQ ID NO: 47 - protein, 4 aa, Homo sapiens
KDEL

SEQ ID NO: 48 - protein, 15 aa, Homo sapiens
LYLSRRSFIDEKKMP

SEQ ID NO: 49 - protein, 6 aa, Simian virus 40
PKKRKV

SEQ ID NO: 50 - protein, 6 aa, Homo sapiens
ARRRRP

SEQ ID NO: 51 - protein, 10 aa, Homo sapiens
EEVQRKRQKL

SEQ ID NO: 52 - protein, 9 aa, Homo sapiens
EEKRKRTYE

SEQ ID NO: 53 - protein, 16 aa, Homo sapiens
KRPAATKKAGQAKKKK

SEQ ID NO: 54 - protein, 12 aa, Homo sapiens
PKKKRKVEDPYC

SEQ ID NO: 55 - protein, 35 aa, Homo sapiens
RPKKRKVRRRFNINDRIKELGTLIPKSNDPDMRWN

SEQ ID NO: 56 - protein, 33 aa, Homo sapiens
RPKKRKVRRRVKRPMNAFMVWAQAARRKLADQY

SEQ ID NO: 57 - protein, 38 aa, Homo sapiens
RPKKRKVRRRKMQQLEQMLTALDQMRRSIVSELAGLLS

SEQ ID NO: 58 - protein, 38 aa, Homo sapiens
RPKKRKVRRRQLMLEPYALDMSRIRVLSESLGLATQSG

SEQ ID NO: 59 - protein, 48 aa, Homo sapiens
TTGGSSPQGLEAKRPKKRKVRRRFNINDRIKELGTLIPKSNDPDMRWN

SEQ ID NO: 60 - protein, 46 aa, Homo sapiens
TTGGSSPQGLEAKRPKKRKVRRRVKRPMNAFMVWAQAARRKLADQY

SEQ ID NO: 61 - protein, 51 aa, Homo sapiens
TTGGSSPQGLEAKRPKKRKVRRRKMQQLEQMLTALDQMRRSIVSELAGLLS

SEQ ID NO: 62 - protein, 51 aa, Homo sapiens
TTGGSSPQGLEAKRPKKRKVRRRQLMLEPYALDMSRIRVLSESLGLATQSG